(19) United States
(12) Patent Application Publication
(10) Pub. No.: US 2022/0064708 A1
(43) Pub. Date: Mar. 3, 2022

(54) METHOD FOR PREPARING LIQUID BIOPSY USING PVA SPONGE

(71) Applicant: General Incorporated Association, Bio Education Laboratory, Osaka (JP)

(72) Inventor: Kenji KINOSHITA, Shizuoka (JP)

(21) Appl. No.: 17/422,880

(22) PCT Filed: Jan. 15, 2020
(86) PCT No.: PCT/JP2020/001064

§ 371 Date: Jul. 14, 2021

(30) Foreign Application Priority Data
Jan. 15, 2019 (JP) 2019-004438
Mar. 29, 2019 (JP) 2019-068152

(51) Int. Cl. C12Q 1/686; C12Q 1/6848; C12Q 1/6851; C12Q 1/6806; G01N 1/12

(52) U.S. Cl. CPC C12Q 1/686; C12Q 1/6848; G01N 1/12; C12Q 1/6806; C12Q 1/6851

(57) ABSTRACT

Provided are: an agent or material for suppressing and reducing reaction inhibition and noise that are caused by a substance in a biological sample; a method using same, for suppressing or reducing reaction inhibition and noise that are caused by a substance in a biological sample; and a method for detection, measurement, or identification of a specific component (e.g., a gene or gene product) in a biological sample using same. The present disclosure pertains to using a PVA sponge and suppressing an amplification activity inhibiting effect on a nucleic acid polymerase caused by a biological sample or a component thereof.

### TECHNICAL FIELD

The present disclosure is directed to preparation of a sample for biological or chemical measurement. More specifically, the present disclosure is directed to sample preparation for gene analysis or drug analysis in a biological sample.

### BACKGROUND ART

In the medical field, analysis of a sample obtained from an organism is required upon studying the effect of a drug. For example, the effect or side effect of a drug can be expected by studying the genetic polymorphism of a drug metabolizing enzyme from a biological sample of a subject before administering the drug. In addition, it is possible to confirm whether a drug exhibits effective kinetics even after administration of the drug by monitoring drug concentration in blood.

However, many contaminants are comprised in a matter obtained from an organism (e.g., blood), which may prevent precise measurement, enzyme reaction necessary for the measurement, and the like. In order to analyze information from a biological sample, it is necessary to go through a complex purification step.

### SUMMARY OF INVENTION

### Solution to Problem

The present disclosure provides a quick sampling method of a biological sample omitting the purification step by using a polyvinyl alcohol (PVA) sponge, and a material for sampling or a kit for sampling used in said method. The inventors found out that the characteristic of the PVA sponge is useful for sampling of a biological sample including liquid biopsy.

A PVA sponge can suppress inhibition of a polymerase amplification inhibitory component in a biological sample, and the present disclosure provides an agent for suppression or a material for suppression comprising a PVA sponge that suppresses polymerase amplification activity inhibiting effect by body fluid (e.g., blood) or a component thereof, a kit comprising the above, or a method using the above. The PVA sponge can capture a microorganism (e.g., cell), and the present disclosure provides a capturing agent or a capturing material comprising a PVA sponge that captures a microorganism or a portion thereof, a kit comprising the above, or a method using the above. In addition, a PVA sponge can be used for stabilization of a nucleic acid, and the present disclosure provides a nucleic acid stabilizing agent or a nucleic acid stabilizing material comprising a PVA sponge, a kit comprising the above, or a method using the above to stabilize a nucleic acid or to preserve a nucleic acid.

For example, the present disclosure provides the following embodiments.

(Item A1) An agent for suppression or a material for suppression that suppresses polymerase amplification activity inhibition effect by body fluid or a component thereof, comprising a PVA sponge.
(Item A1-1) The agent for suppression or the material for suppression of any of the above item, wherein the PVA sponge has an average pore size of 80 μm to 200 μm.
(Item A1-2) The agent for suppression or the material for suppression of any of the above items, wherein the PVA sponge has a porosity of 89% to 91%.
(Item A1-3) The agent for suppression or the material for suppression of any of the above items, wherein the body fluid is blood or saliva.
(Item A2) An agent for sampling or a material for sampling a liquid biopsy, comprising the agent for suppression or the material for suppression of any of the above items.
(Item A3) A kit comprising the agent for sampling or the material for sampling of any of the above items.
(Item A4) The kit of any of the above items, which is for nucleic acid analysis.
(Item A5) The kit of any of the above items, wherein the nucleic acid analysis comprises the step of amplification of a nucleic acid.
(Item A6) The kit of any of the above items, which is for analysis of a circulating nucleic acid.
(Item A7) The kit of any of the above items, wherein the circulating nucleic acid is a tumor circulating DNA or a tumor circulating miRNA.
(Item A8) The kit any of the above items, which is for monitoring of tumor amount in a body, determination of agent resistant gene, early detection of cancer, or recurrence monitoring.
(Item A9) The kit of any of the above items, wherein the circulating nucleic acid is cfDNA.
(Item A10) The kit of any of the above items, which is for prenatal diagnosis.
(Item A11) The kit of any of the above items, which is for detection of chromosomal abnormality or neonatal screening.
(Item A12) The kit of any of the above items, which is for detection of virus infection.
(Item A13) The kit of any of the above items, which is for absolute quantification of a nucleic acid.
(Item A14) The kit of any of the above items, wherein the absolute quantification is carried out by digital PCR.
(Item A15) A method of suppressing polymerase amplification activity inhibition effect by body fluid or a component thereof, encompassing the step of contacting the body fluid or the component thereof with a PVA sponge.
(Item A16) The method of the above item, wherein the body fluid is blood or saliva.
(Item B1) A capturing agent or a capturing material that captures a microorganism or a portion of a microorganism, comprising a PVA sponge.
(Item B1-1) The capturing agent or the capturing material of the above item, wherein the PVA sponge has an average pore size of 80 μm to 200 μm.
(Item B1-2) The capturing agent or the capturing material of any of the above items, wherein the PVA sponge has a porosity of 89% to 91%.
(Item B2) The capturing agent or the capturing material of any of the above items, which is for cell culturing.
(Item B3) The capturing agent or the capturing material of any of the above items, which is for sampling of liquid biopsy.
(Item B4) The kit of any of the above items, which is for nucleic acid analysis.
(Item B5) The kit of any of the above items, wherein the nucleic acid analysis comprises the step of amplification of a nucleic acid.
(Item B6) The kit of any of the above items, which is for capturing of a blood circulating tumor cell.
(Item B7) The kit of any of the above items, which is for prognosis prediction of cancer or re-examination of a drug therapy method.
(Item B8) The kit of any of the above items, which is for quantification of tumor cell number using digital PCR.
(Item B9) The kit of any of the above items, wherein the nucleic acid analysis comprises digital PCR.
(Item B10) The kit of any of the above items, which is for absolute quantification of a bacterium or a virus in an oral cavity or in blood.
(Item B11) The method of the above item that captures a microorganism or a portion of a microorganism.
(Item B13) A method of quantitatively analyzing or capturing a microorganism, comprising:
(a) the step of contacting a constant volume of PVA sponge with a specimen comprising a microorganism which is an analysis or capturing target;
(b) the step of separating the microorganism or a component thereof from the constant volume of PVA sponge; and
(c) the step of analyzing the microorganism or the component thereof as needed.
(Item C1) A nucleic acid stabilizing agent or a nucleic acid stabilizing material, comprising a PVA sponge.
(Item C1-1) The nucleic acid stabilizing agent or the nucleic acid stabilizing material of the above item, wherein the PVA sponge has an average pore size of 80 μm to 200 μm.
(Item C1-2) The nucleic acid stabilizing agent or the nucleic acid stabilizing material of any of the above items, wherein the PVA sponge has a porosity of 89% to 91%.
(Item C2) A kit, which comprises the nucleic acid stabilizing agent or the nucleic acid stabilizing material of any of the above items.
(Item C3) The kit of any of the above items, which is for preservation of a nucleic acid derived from blood.
(Item C4) The kit of any of the above items, which is for separating plasma from blood for preservation.
(Item C5) A method for stabilizing or preserving a nucleic acid, encompassing the step of contacting a PVA sponge with the nucleic acid.
(Item D1) A kit for sampling liquid biopsy, comprising a PVA sponge, characterized by being used in a method comprising:

the step of contacting the PVA sponge with liquid biopsy;

the step of separating a nucleic acid specimen from the PVA sponge in water to obtain supernatant; and

the step of subjecting the supernatant to gene analysis.

(Item D2) The kit of the above item, wherein the method further comprises the step of drying the PVA sponge after the step of contacting.
(Item D3) The kit of any of the above items, wherein the step of obtaining the supernatant comprises heating a PVA sponge in water.
(Item D4) A method for sampling liquid biopsy, comprising:

the step of contacting a PVA sponge with liquid biopsy;

the step of separating a nucleic acid specimen from the PVA sponge in water to obtain supernatant; and

the step of subjecting the supernatant to gene analysis.
(Item D5) The method of the above item, further comprising the step of drying the PVA sponge after the step of contacting.
(Item D6) The method of any of the above items, wherein the step of obtaining the supernatant comprises heating a PVA sponge in water.
(Item E1) A kit for sampling liquid biopsy, comprising a PVA sponge, characterized in being used in a method comprising:

the step of contacting the PVA sponge with liquid biopsy;

the step of separating a nucleic acid specimen from the PVA sponge in water to obtain supernatant;

the step of freeze-drying the supernatant; and

the step of dissolving the freeze-dried supernatant in water.

(Item E2) The kit of any of the above items, wherein the method further comprises the step of drying the PVA sponge after the step of contacting.
(Item E3) The kit of any of the above items, wherein the step of obtaining the supernatant comprises heating a PVA sponge in water.
(Item E4) A method for sampling liquid biopsy, comprising:

the step of contacting a PVA sponge with liquid biopsy;

the step of separating a nucleic acid specimen from the PVA sponge in water to obtain supernatant;

the step of freeze-drying the supernatant; and

the step of dissolving the freeze-dried supernatant in water.

(Item E5) The method of any of the above items, further comprising the step of drying the PVA sponge after the step of contacting.
(Item E6) The method of any of the above items, wherein the step of obtaining the supernatant comprises heating a PVA sponge in water.
(Item F1) A kit for sampling dry attached blood, comprising a PVA sponge, characterized in being used in a method comprising:

the step of contacting the PVA sponge with a dry attached blood;

the step of separating a nucleic acid specimen from the PVA sponge in water to obtain supernatant; and

the step of subjecting the supernatant to gene analysis.

(Item F2) The kit of the above item, wherein the step of obtaining the supernatant comprises heating a PVA sponge in water.
(Item F3) A method for sampling dry attached blood, comprising:

the step of contacting with dry attached blood with a PVA sponge;

the step of separating a nucleic acid specimen from the PVA sponge in water to obtain supernatant; and

the step of subjecting the supernatant to gene analysis.

(Item F4) The method of the above item, wherein the step of obtaining supernatant comprises heating a PVA sponge in water.
(Item G1) An agent for sampling or a material for sampling comprising a PVA sponge, which is for analysis of circulating nucleic acid in plasma.
(Item G1-1) The agent for sampling or the material for sampling the above item, comprising one or more of the characteristics of any of the above items.
(Item G2) The agent for sampling or the material for sampling the above item, which is for analysis of a circulating nucleic acid in plasma.
(Item G3) The kit of any of the above items, wherein the analysis of the circulating nucleic acid in plasma is quantitative analysis.
(Item G4) The kit of any of the above items, wherein the circulating nucleic acid is a tumor circulating nucleic acid.
(Item G5) A method of carrying out circulating analysis in plasma, comprising:

the step of contacting a PVA sponge with plasma; and

the step of subjecting the PVA sponge to gene analysis.

(Item G5-1) The method of the above item, comprising one or more of the characteristics of any of the above items.
(Item G6) A kit for analysis of a circulating nucleic acid in plasma, comprising a PVA sponge, characterized in being used in a method comprising:

the step of contacting the PVA sponge with plasma; and

the step of subjecting the PVA sponge to gene analysis.

(Item G6-1) The kit of the above item, comprising one or more of the characteristics of any of the above items.

In the present disclosure, one or more of the above-described features is or are intended to be able to be provided in the explicitly shown combination as well as in further combination. Those skilled in the art would recognize further embodiments and advantages of the present disclosure when the following detailed explanation is read and understood as needed.

### Advantageous Effects of Invention

Sampling using a PVA sponge can be carried out quickly, easily, and with low costs. The shortened step (including reduction of steps and shortening of time, or the like) can increase reliability and reduce labor cost. In addition, difference in DNA quality between samples is minimized. A PVA sponge can be used for absolute quantification, wherein the absolute quantification is significant when carrying out a plurality of measurements (e.g., monitoring of a subject). In addition, after drying of a PVA sponge, a sample can be preserved and transported at room temperature, wherein the sample can also be easily handled.

### BRIEF DESCRIPTION OF DRAWINGS

FIG. 1 is a diagram showing the result of nucleic acid amplification in a sample of when using THUNDERBIRD ProbeqPCR Mix in Example 1A. Each numeral shows the number of DNA copies in each sample. S refers to what is collected with a PVA sponge, and M refers to what is collected with a water-soluble paper. The one without an alphabet refers to a control DNA in a solution.

FIG. 2 is a diagram showing the result of nucleic acid amplification in a sample of when using TaqPath qPCR Master Mix in Example 1A. Each numeral shows the number of DNA copies in each sample. S refers to what is collected with a PVA sponge, and M refers to what is collected with a water-soluble paper. The one without an alphabet refers to a control DNA in a solution.

FIG. 3 is a diagram showing a method of sampling in Example 1B and the result of nucleic acid amplification in a sample sampled by the sampling method.

FIG. 4 is a diagram showing a method of sampling in Example 1C and the result of nucleic acid amplification in a sample sampled by the sampling method.

FIG. 5 is a diagram showing a method of sampling in Example 1D and the result of nucleic acid amplification in a sample sampled by the sampling method.

FIG. 6 is a schematic diagram of the serum gathering device of the present disclosure and one example of a method using the serum gathering device.

FIG. 7 is a schematic diagram of the serum gathering device of the present disclosure and one example of a method using the serum gathering device.

FIG. 8 is a diagram showing the result of amplification of a nucleic acid derived from a white blood cell in a PVA disk in Example 2A. Each number is the time of incubation in the peripheral blood of a PVA disk.

FIG. 9 is a diagram showing the result of nucleic acid amplification of Example 2B. The result of PVA sponge wash supernatant PCR gene amplification is shown as Ds, Es, Fs and Ys, and the result of PBS wash PVA sponge disk treated supernatant PCR gene amplification is shown as Ds, Ed, Fd and Yd.

FIG. 10 is a diagram showing the result of nucleic acid amplification of Example 2C. Each liquid amount corresponds to the result of amplification of a nucleic acid derived from the shown amount of whole blood dropped on a PVA disk.

FIG. 11 is a schematic diagram showing a method of capturing cell in saliva using a PVA sponge in Example 2D.

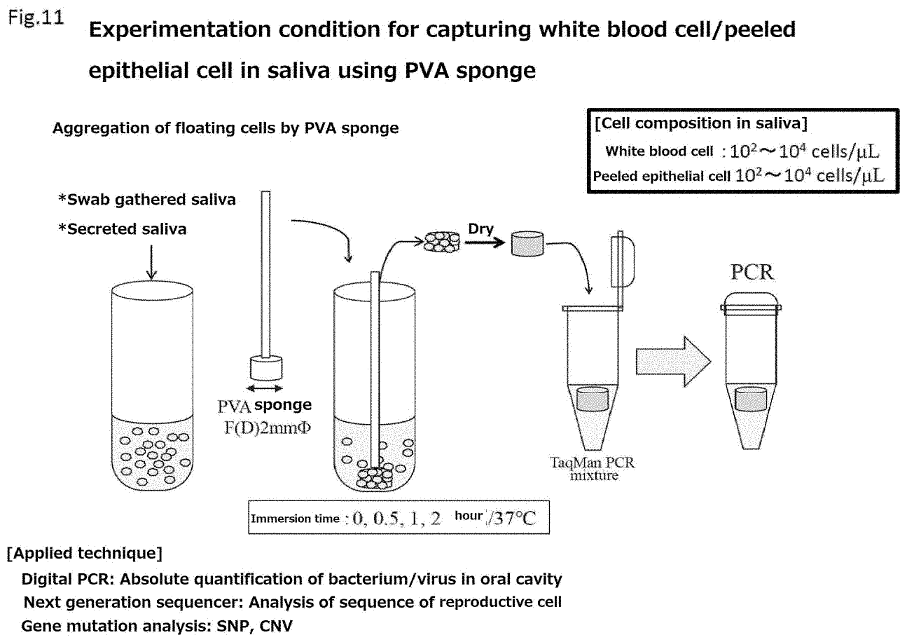

FIG. 12 is a diagram showing the result of amplification of a nucleic acid derived from a cell of saliva captured by a PVA sponge in Example 2D.

FIG. 13 is a diagram showing the result of amplification of a nucleic acid derived from a sample directly sampled from an oral cavity by a PVA sponge in Example 2E.

FIG. 14 is a schematic diagram showing the saliva gathering device of the present disclosure and one example of a method using the device.

FIG. 15 is a schematic diagram showing the cancer cell gathering device of the present disclosure and one example of a method using the device.

FIG. 16 is a schematic diagram showing one example of a method carrying out prompt gene examination of a solid tumor cell utilizing a PVA sponge.

FIG. 17 is a schematic diagram showing one example of a method carrying out agent selectivity test of a solid tumor cell utilizing a PVA sponge.

FIG. 18 is a schematic diagram of a method of DNA stability test in serum in Example 3.

FIG. 19 is a diagram showing the result of genome DNA stability test in Example 3.

FIG. 20 is a diagram showing the result of plasmid DNA stability test in Example 3.

FIG. 21 is a diagram showing the procedure and result of the freeze-drying experimentation of serum treated PVA sponge adjustment liquid in Example 4.

FIG. 22 is a diagram showing each specimen (right panel) and the result of amplification or a nucleic acid derived from blood wiped off from the specimens with a PVA sponge (left panel).

FIG. 23 is a schematic diagram showing an example of a post-surgery early recurrence diagnosis using the present disclosure.

FIG. 24 is a schematic diagram showing an example of therapeutic effect monitoring using the present disclosure.

FIG. 25 is a diagram showing the procedure and result of direct quantification of cfDNA in plasma using a PVA sponge in Example 7A.

FIG. 26 is a diagram showing the procedure and result of the direct quantification of cfDNA in plasma using a PVA sponge in Example 7B.

### DESCRIPTION OF EMBODIMENTS

The present disclosure is explained hereinafter while showing the best form. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

The definition and/or basic technical content of the terms especially used herein is appropriately explained below.

### PVA Sponge

The present disclosure provides a method for sampling using a polyvinyl alcohol (PVA) sponge and a material for sampling or a kit for sampling used in said method.

As used herein, “sampling” refers to extraction of a specimen (sample) in an examination method such as liquid biopsy, and an agent and a material therefor are referred to as agent for sampling and material for sampling, respectively.

As used herein, “PVA sponge” is a porous material with polyvinyl alcohol crosslinked, wherein the structure and production method thereof are known in the subject technical field (see Japanese Laid-Open Publication No. 2001-302840 and the like). Crosslinking can be carried out with formaldehyde or the like.

As used herein, “polyvinyl alcohol (PVA)” refers to any polymer with vinyl alcohol <rational formula (—CH₂CH(OH)—)ₙ> polymerized, which is a water-soluble polymer having a hydroxyl group which is obtained by hydrolyzing polyvinyl acetate with acid or alkali. The polymer is also called poval or PVOH. Any PVA can be used for manufacturing a PVA sponge.

A PVA sponge can be manufactured in, for example, the following manner. That is, PVF (polyvinyl formal) which is an insoluble matter is manufactured by a formalization reaction which binds formaldehyde to water-soluble PVA (polyvinyl alcohol, poval) while using acid as a catalyst, in which step a pore generating agent is added and formation of a pore is carried out to extract this pore generating agent after completion of insoluble PVF, wherein the completed PVF becomes a porous body to form three-dimensional dendritic network continuous pores. This porous body is a PVA sponge.

Almost 90% of the volume of a PVA sponge can consist of void, but the PVA sponge can still maintain a structural strength that enables use as a base material. In addition, a substrate forming this void (pore) establishes a three-dimensional network structure, wherein individual pores are all continued. Such an integral structure is the greatest feature of a PVA sponge, which allows numerous functions. In addition, unlike a common urethane sponge or the like, a PVA sponge has extremely high hydrophilicity and causes a capillary phenomenon by fine pores spreading in all directions, thus having a very excellent water absorbing property/water retaining property. In addition, a PVA sponge has flexibility/elasticity in a wet state, wherein a surface of an object can be prevented from being damaged especially when used as a washing material or a wiping material.

A commercially available PVA sponge (e.g., made by AION Co., Ltd.) can be used. Regarding the parameters of a PVA sponge, the pore size (average pore size) or porosity of the sponge can be selected. Porosity is the proportion of the volume of the void to the volume of the sponge.

As used herein, “average pore size” is an (arithmetic) average value of a pore size, which can be measured based on JIS Z 8831-2.

As used herein, “porosity” is the ratio of the volume of the fine pore and void capable of adsorption to the entire volume occupied by a predetermined amount of solid body, which can be measured based on JIS Z 8831-2.

A table showing the physical properties of a PVA sponge provided by AION Co., Ltd. is described below. When a PVA sponge is referred to by the article number below in other portions herein, the PVA sponge of the corresponding article number made by AION Co., Ltd. is referred to.

TABLE 1
Basic physical property

| Item/article | | | | | | | |
|---|---|---|---|---|---|---|---|
| number | D(D) | E(D) | F(D) | Y(D) | EB(D) | FB(D) | GB(D) |
| Average pore | 80 | 130 | 200 | 180 | 150 | 300 | 700 |
| size (μm) | | | | | | | |
| Porosity (%) | 89 | 90 | 91 | 90 | 89 | 91 | 90 |
| Tensile strength | 500 | 430 | 370 | 430 | 280 | 250 | 230 |
| (kPa)*1 | | | | | | | |
| Tensile strength | 250 | 250 | 250 | 250 | 200 | 300 | 200 |
| (%)*2 | | | | | | | |
| 30% compression | 6.9 | 4.7 | 3.0 | 6.3 | 4.3 | 1.5 | 4.1 |
| stress (kPa)*3 | | | | | | | |
| Water retention | 1000 | 1100 | 1300 | 1050 | 1050 | 1300 | 600 |
| rate | | | | | | | |
| Heat resistance | 60 | 60 | 60 | 60 | 60 | 60 | 60 |
| (° C.) | | | | | | | |

Since FB and GB have large pore sizes, it can be considered that a PVA sponge of a different standard is more suitable for sampling of a biological specimen. In one preferable embodiment of the present disclosure, the pore size of a PVA sponge is about 80 μm to about 200 μm.

In one preferable embodiment of the present disclosure, the porosity of a PVA sponge is 89% co 91%.

It is considered that the PVA sponges of D(D) to EB(D) do not have much of a difference in terms of capacity in sampling of a biological specimen. It is considered that F(D) is the easiest to handle in view of the mechanical characteristics.

In one aspect, the present disclosure provides an agent for suppression or a material for suppression that suppresses polymerase amplification activity inhibition effect by body fluid or a component thereof, comprising a PVA sponge. Although not wishing to be bound by any theory, this usage is due to the inventor's discovery of an amplification inhibiting matter in blood being trapped to be able to avoid inhibition in the amplification reaction thereafter by adsorbing the blood to a PVA sponge. Said trapping of an amplification inhibiting matter is considered effective for any body fluid comprising such an amplification inhibiting matter. Therefore, aside from an agent for suppression or a material for suppression that suppresses amplification activity inhibition effect, such a property may provide a blood sampling kit comprising a PVA sponge, a sampling method using the blood sampling kit, detection (postoperative monitoring) of ctDNA derived from a cancer cell in serum (plasma), detection of tumor marker circulating miRNA in serum (plasma), detection (prenatal diagnosis) of floating DNA (cfDNA) in serum (plasma), whole blood genome DNA detection (neonatal screening), monitoring (predisease) of cancer marker gene in serum (plasma), possibility of absolute quantification (real time/digital PCR), whole blood genome DNA detection (neonatal screening: presence/absence of a genetic disease) and the like.

As used herein, polymerase “amplification activity inhibition (effect)” refers to the concept of the amplification activity normally possessed by polymerase being inhibited, i.e., being reduced compared to the normal activity or being lost. As used herein, “suppression” of the “amplification activity inhibition effect” of polymerase also encompasses the concept of more activity than the normally possessed amplification activity being recovered in addition to the concept of this “amplification activity inhibition effect” being suppressed, i.e., the degree of inhibition being reduced or being lost (i.e., recovery to the normally possessed amplification activity).

The inventor found cut that a PVA sponge captures a cell such as a white blood cell in saliva. Such a property may provide possibility of capturing/culturing of a cell in blood, detection of RNA/DNA of a virus/bacterium in serum, absolute quantification of oral bacterium/virus with digital PCR from saliva sampling, and comparison of the number of copies between a normal cell and a cancer cell with digital PCR from cancer cell sampling. In addition, since a PVA sponge can retain a certain number of microorganisms (cells or the like) per volume, a PVA sponge may enable quantitative sampling without depending on the mount of microorganisms in a sample.

In addition, a nucleic acid can be stabilized by adsorbing a specimen to a PVA sponge, which is also useful for preservation/transportation after sampling.

A sample for biological or chemical analysis of a biological sample can be prepared by dropping and drying the biological sample (e.g., blood) on a PVA sponge. In one embodiment, the biological analysis is gene analysis.

### Specimen

“Nucleic acid-containing biological material” used herein refers to any biological material comprising a nucleic acid which would become a specific target, which comprises biological tissue/cell (e.g., a cell of a plant or an animal), microorganism such as bacterium and virus and the like as well as a preparation derived therefrom and the like. A “specimen comprising a nucleic acid-containing biological material” can include biological specimen or clinical specimen such as nasal discharge, nasal swab, ocular conjunctival swab, pharyngeal swab, expectoration, stool, blood, serum, plasma, cerebrospinal fluid, saliva, urine, sweat, milk, semen, oral swab, interdental swab, wet earwax, vaginal swab and cell tissue, as well as food product and the like. A specimen to be examined may include a specimen encompassing an RNA selected from the group consisting of a cell, fungus, bacterium and virus. A gene that would become a target for amplification or a specific gene that would become a target for detection may be a DNA or an RNA. In this case, the present disclosure directly adds a specimen to be examined carried by a PVA carrier to a reaction liquid and carries out an RNA amplification reaction to enable direct amplification of RNA existing in said specimen to be examined. In this regard, “direct addition” means that the process of extracting an RNA from a specimen to be examined encompassing this RNA prior to RNA amplification is riot required.

### Liquid Biopsy

As used herein, “liquid biopsy” refers to a liquid biological sample, including samples of body fluids such as blood, urine, saliva and semen. While analysis of liquid biopsy can carry out examination with low invasiveness compared to conventional biopsy gathering, there are many cases in which detection must be carried out with higher sensitivity than before (e.g., 100 to 1,000-fold). This is because analysts of liquid biopsy would be analysis of a biomolecule that leaked out in a liquid. For example, analysis of liquid biopsy includes analysis of a circulating cancer cell (CTC), circulating abnormal cell (CAG), stem cell, circulating cancer gene (CTG), cell-free gene (cfDNA), microRNA (miRNA), trace amount of molecule in blood, peptide, microparticle, or the like.

As used herein, “microorganism” is a particle encapsulating a molecule having gene information, which refers to any particle that can be duplicated (regardless of the possibility on its own). The “microorganism” herein encompasses a unicellular organism, bacterium, cell derived from a multicellular organism, fungus, virus and the like.

### Gene Analysis

As used herein, “gene analysis” refers to the concept of studying the state of a nucleic acid (DNA, RNA, or the like) in a biological sample. In one embodiment, gene analysis can include a gene analysis that utilizes a nucleic acid amplification reaction. In addition to the above, examples of gene analysis can include sequencing, genotyping/polymorphism analysis (SNP analysis, copy number variation, restriction fragment length polymorphism, repeat number variation), expression analysis, Quenching Probe (Q-Probe), SYBRgreen method, melting curve analysis, real-time PCR, quantitative RT-PCR, digital PCR and the like. One example of gene analysis is determination of genotype using the TaqMan probe method. The Quenching Probe method is described in https://www.aist.go.jp/Portals/0/resource_images/aist_j/aistinfo/aist_today/vol08_2/vol08_02_p18_p19.pdf and the like.

An amplification reaction of a nucleic acid is generally involved in such a gene analysis. When a biological sample (e.g., blood or saliva) is directly added to a reaction liquid of a nucleic acid amplification reaction (e.g., PCR), there is a case in which the action caused by a matter in the biological sample causes inhibition of the amplification reaction (e.g., DNA polymerase such as Taq DNA polymerase). Inhibition of said amplification reaction is suppressed with a sample prepared by dropping a biological sample (e.g., blood or saliva) on a PVA sponge. Although not to be bound by any theory, it is considered that this is because a PVA sponge has the ability to remove matters (heme, polysaccharides, polyphenol, fulvic acid, pigment, ion and the like) that inhibit PCR amplification.

### Polymerase

A nucleic acid polymerase is used in a nucleic acid amplification reaction that may be used in one embodiment of the present disclosure. In one embodiment, any DNA polymerase can be used as a DNA polymerase that is used for gene analysis without any particular restriction as long as the DNA polymerase is a polymerase that synthesizes a nucleic acid by primer addition and has excellent heat resistance, typically Taq DNA polymerase. The nucleic acid amplification reaction particularly utilized in the present disclosure uses a polymerase having 5′→3′ exonuclease activity. Although not wishing to be bound by any theory, this is because, while the use of a KOD polymerase is a complicated method since it is not possible to carry out detection with a label like a DNA polymerase due to a difference in the amplification method where there would be no choice but to carry out analysis with a cut pattern with a restriction enzyme, the use of a polymerase having 5′→3′ exonuclease activity like Taq DNA polymerase can realize simple analysis. A Family A (Pol I-type) DNA polymerase is included in the polymerase having 5′→3′ exonuclease activity.

Such a DNA polymerase can include, for example, Taq DNA polymerase derived from Thermos aquaticus, as well as Tth DNA polymerase, or a mixture of at least any of the above-mentioned DNA polymerases, and the like. In addition, since Tth DNA polymerase and a C. therm DNA polymerase derived from Carboxydothermus hydrogenoformans have RT activity, the polymerases have the feature of being able to manage with one type of enzyme upon carrying out RT-PCR with One tube-One step. In one embodiment, while a method using other polymerases may be utilized, the direct use of a sample for a polymerase having 5′→3′ exonuclease activity like Taq DNA polymerase is recommended as a simple method. It was discovered in the present disclosure that amplification inhibition comprised in a biological sample (e.g., blood sample) is caused in such a case. Such problem of amplification inhibition comprised in a biological sample (e.g., blood sample) was unexpectedly solved by using a PVA sponge like the present disclosure. This allows direct application of a biological sample such as a blood sample to an analysis method (e.g., so-called Taqman method or a method equivalent thereto) using a polymerase having 5′→3′ exonuclease activity. Such an achievement was not possible with conventional technique.

A reaction using a polymerase can include, for example, PCR (Polymerase Chain Reaction) method, LAMP (Loop-Mediated Isothermal Amplification) method, SDA (Strand Displacement Amplification) method, RT-SDA (Reverse Transcription Strand Displacement Amplification) method, RT-PCR (Reverse Transcription Polymerase Chain Reaction) method, RT-LAMP (Reverse Transcription Loop-Mediated Isothermal Amplification) method, NASBA (Nucleic Acid Sequence-Based Amplification) method, TMA (Transcription Mediated Amplification) method, RCA (Rolling Cycle Amplification) method, ICAN (Isothermal and Chimeric primer-initiated Amplification of Nucleic acids) method, UCAN method, LCR (Ligase Chain Reaction) method, LDR (Ligase Detection Reaction) method, SNAP (Smart Amplification Process) method, SMAP2 (Smart Amplification Process Version 2) method, PCR-Invader method, Multiplex PCR-Based Real-Time Invader Assay (mPCR-RETINA), Quenching Probe (Q-Probe) and the like. Preferably, a so-called Taqman method is used. While the buffer is not particularly restricted, it is possible to use a product that does not weaken the effect of removal of amplification inhibition among EzWay™ (KOMA Biotechnology), Ampdirect® (Shimadzu Corporation), Phusion® Blood Direct PCR kit buffer (New ENGLAND Bio-Labs), MasterAmp® PCR kit (made by Epicentre) and the like.

The primer used in the reaction in the present disclosure can be designed with an appropriate known method at the time point of determining a gene that would become a target for amplification or a specific gene that would become a target for detection. The primer used in the reaction in the present disclosure is not particularly restricted as long as the primer can specifically amplify a gene that would become a target for amplification or a specific gene that would become a target for detection.

It is preferable that the amplification of a gene comprised in a specimen to be examined or the detection of a specific gene comprised in a specimen to be examined in the present disclosure is carried out on a plate-like or tube-like insoluble carrier. Such an insoluble carrier can include a plastic that is insoluble to a reaction liquid, a tube made of glass or the like, as well as a 96 hole well and the like. Furthermore, tube-like refers to a hollow state, which may be a form of a PCR tube with a bottom or an Eppendorf tube.

Specifically, a reaction liquid is first introduced to a plate-like or tube-like insoluble carrier. In the, case of a tube-like insoluble carrier, a buffer, polymerase and a reaction liquid containing a reagent such as primer are introduced, and in case of a plate-like insoluble carrier, the reaction liquid is placed on the surface thereof. In addition, the disposition is arranged so that the reaction liquid directly contacts a specimen to be examined and a PVA sponge, wherein an amplification method such as the above-mentioned PCR method is carried out.

The quantitative PCR used herein can be carried out using any known method such as a known real-time PCR. These methods are methods that detect DNA amplification amount in real tame using a label such as a fluorescence reagent, which typically require an apparatus integrating a thermal cycler and a spectrofluorometer. Such an apparatus is commercially available. There are several methods depending on the fluorescence reagent to be used, which include, for example, intercalator method, TaqMan™ probe method, Molecular Beacon method and the like. All are methods which add a fluorescence reagent or a fluorescence probe called an intercalator, TaqMan™ probe, or Molecular Beacon probe to a PCR reaction system comprising a primer pair for amplification of a genome region comprising a template genome and a target SNP site. In the TaqMan™ probe method (also referred to as TaqMan™ method) which is preferably used in the present disclosure, a TaqMan™ probe is an oligonucleotide that may hybridize to an amplification region of a target nucleic acid wherein both ends are modified with each of a fluorescence matter and a quenching matter, which hybridizes a target nucleic acid upon annealing but cannot emit fluorescence due to the presence of the quenching matter, but emits fluorescence by being dissolved by the exonuclease activity of a DNA polymerase upon an extension reaction to free the fluorescence matter. Therefore, the produced amount of amplification product can be monitored by measuring the fluorescence strength, which enables estimation of the original template DNA amount.

Copy number analysis using a Taqman assay is, for example, a method that is known in the subject field. A Taqman assay is based on a 5′-nuclease assay that is also called a fluorogenic 5′-nuclease assay; wherein Holland et al., Proc. Natl. Acad. Sci. USA 88:7276-7280 (1991); and Held et. al., Genome Research 6:986-994 (1996) can be referred to.

In the procedure of TaqMan PCR, two oligonucleotide primers are used to make an amplicon specific to a PCR reaction. A third oligonucleotide (TaqMan probe) is designed to hybridize with a nucleotide sequence in an amplicon positioned between two PCR primers. A probe may have a structure that cannot be extended by a DNA polymerase used in a PCR reaction, and is normally (but not essential) co-labeled by a fluorescence reporter dye and a quenching agent portion which are in close contact to one another. Light emission from a reporter dye is quenched by a quenching portion when a fluorescent body and a quenching agent are in close contact as they are on the probe. In some cases, the probe may be labeled only by a fluorescence reporter dye or a different detectable portion.

A thermally stable DNA-dependent DNA polymerase having 5′-3′ nuclease activity is used in a TaqMan PCR reaction. During a PCR amplification reaction, the 5′-3′ nuclease activity of a DNA polymerase causes cleavage of a label probe hybridizing with an amplicon in a template-specific manner. The probe fragment which is obtained dissociates from a primer/template complex and then a reporter dye would be released from the quenching effect of the quenching agent portion. About one molecule of reporter dye is freed for each newly synthesized amplicon molecule and an unquenched reporter dye is detected to provide a quantitatively interpreted base of data in the form of the amount of a released fluorescence reporter dye being in direct proportion to the amount of an amplicon template.

One scale of TaqMan assay data is normally expressed as a threshold cycle (CT). The fluorescence level is recorded during each PCR cycle and is in proportion to the amount of the product that has been amplified by an amplification reaction up to that point. The PCR cycle upon the initial recording wherein a fluorescence signal has been determined to be statistically significant, or of when a fluorescence signal exceeds a different some sort of arbitrary level (e.g., arbitrary fluorescence level (AFL)), is the threshold cycle (CT). A protocol or reagent for a 5′-nuclease assay is known to those skilled in the art and is described in various reference documents. For example, U.S. Pat. No. 6,214,979 (Gelfand et al.); U.S. Pat. No. 5,804,375 (Gelfand et al.); U.S. Pat. No. 5,487,972 (Gelfand et al.); Gelfand et al., U.S. Pat. No. 5,210,015 (Gelfand et al.) and the like can be referred to regarding a nuclease reaction and probe.

TaqMan™ PCR can be carried out using a commercially available kit and apparatus, which are, for example, ABI PRISM® 7700 Sequence Detection System (Applied Biosystems, Foster City, Calif., USA), Light Cycler® (Roche Applied Sciences, Mannheim, Germany) and the like. In a preferable embodiment, the 5′-nuclease assay procedure is carried out on, but not limited to, a real-time quantitative PCR apparatus such as the ABI PRISM® 7700 Sequence Detection System. This system typically consists of a thermocycler, a laser, a charge coupled device (CCD), a camera and a computer. This system amplifies a sample in a microtiter plate format such as a 96 well on a thermocycler. During the amplification, a laser induction fluorescence signal is collected through an optical fiber in real time regarding all wells and detected with a CCD camera. This system comprises a software for operation of a device and analysis of data.

For example, a Taqman assay, which has been reported regarding CYP2D6, can be used as another example of the specific procedure of the Taqman method (Bodin et al., J Biomed Biotechnol. 3: 248-53 2005). In this case, a new reverse primer can be designed by Primer Express or the like and the copy number analysis can be performed again in order to obtain accurate data as needed. A Taqman probe, wherein a 5′ terminus is labeled with FAM and No Fluorescence Quencher and MGB are coupled to a 3′ terminus, can be used. A RNase P assay (ThermoFisher) labeled with an appropriate label (e.g., VIC) can be used as a reference gene. All Taqman assays can be performed in accordance with a reported protocol that can be obtained from a manufacturer and the copy number calculation can be performed by the ΔΔCt method (Bodin et. al., 2005). As one example, assuming that a sample having a median ΔCt value has two copies, the sample can be used as, but not limited to, a calibrator. It is possible to test all samples in duplicate and use a mean copy number value in scatter plot analysis, but the sample number can be increased or decreased as needed.

TaqPath™ ProAmp™ Master Mix can be used as a real-time PCR reagent in the present disclosure. The advantage of TaqPath™ ProAmp™ Master Mix includes extraordinary data quality (high specificity, dynamic range and reproducibility can be provided in genotyping and copy number variation (CNV) analysis even in the presence of a PCR inhibiting matter) and resistance against a PCR inhibiting matter (capable of handling a prepared sample derived from a human or an animal (buccal swab, blood and card punch)). When combined with the suppression of the amplification activity inhibition effect by blood or a component thereof of the PVA sponge in the present disclosure, it is possible to subject blood to a reaction without carrying out any extraction or purification. For example, a PVA sponge contacted with liquid biopsy (blood, serum, plasma, saliva, or the like) can be added to a reaction liquid. In the present disclosure, the step of obtaining supernatant or plasma from a PVA sponge can be the step of directly subjecting the PVA sponge to the following reaction. After contacting with the liquid biopsy, the PVA sponge can be dried as needed.

In a digital PCR, a mixture of nucleic acids is distributed to many reaction wells so that while a certain well would comprise one target molecule, a different well would not comprise a target. A normal PCR is performed at each reaction liquid to identify a well that does not comprise a target molecule. Correction calculation can be carried out with a standard statistic model to obtain the final concentration value. Since a Ct value is not used for quantification of copy number in a digital PCR, comparison with a known standard not required in absolute quantification.

### Target of Analysis

As used herein, “circulating tumor cell (CTC)” refers to a cell that is freed from a primary tumor tissue or a metastatic tumor tissue into blood through epithelial-mesenchymal transition (EMT) to circulate in blood stream. After being freed from the primary tumor site, the CTC circulates within blood to invade other organs and form a metastatic tumor (metastatic lesion). Since a CTC exists in peripheral blood of a cancer patient, detection thereof enables determination of the stage of metastases to contribute to predicting the prognosis of therapy.

As used herein, “cfDNA (cell-free DNA)” refers to a free DNA that has been released from a cell due to cell death in blood, wherein a DNA derived from a cancer cell is called a “circulating tumor DNA (ctDNA)”.

ctDNA is currently used as a clinically useful tumor marker for monitoring of the amount of tumor in a body, determination of an agent-resistant gene, super early cancer discovery and the like. A serum tumor marker is often used today as the simplest tool for monitoring of the amount of tumor in a body. In fact, a tumor marker is a useful diagnosis assisting tool when recurrence has advanced to a certain degree or when a change in time series reflects therapy. Super early cancer discovery, which holds the possibility to exert power upon cancer screening or the like, holds the possibility of being able to provide highly precise cancer screening only by collecting blood. There are also problems such as restriction regarding blood gathering and maturation of the technique for accurately measuring super low frequency mutation that may be as low as 0.1% or lower. However, it is possible that every day cancer diagnosis would gradually change from “seeing” to “measuring” in the future by ctDNA.

However, in order to use an unstable ctDNA research as a standard technique, there are problems to be solved such as centrifugation of plasma within two hours after collecting blood, minimization of bias among patents and among experimenters quality control/quantification for bias correction, simplification of gene examination process, and enablement of absolute quantification and not relative quantification with respect to a control. Sampling using a PVA sponge is quick, easy and inexpensive, and is useful to solve the above-mentioned problems by enabling high reliability and reduction of labor cost by step reduction, minimization of DNA quality difference among samples, absolute quantification (essential for monitoring), and preservation and transport of a sample at room temperature after drying.

### Preferable Embodiment

One aspect of the present disclosure provides an agent for suppression or a material for suppression that suppresses polymerase amplification activity inhibition effect by body fluid (e.g., blood or saliva) or a component thereof, comprising a PVA sponge. In this aspect, the present disclosure also provides a method of suppressing polymerase amplification activity inhibition effect by body fluid (e.g., blood or saliva) or a component thereof, encompassing the step of contacting the body fluid or the component thereof with a PVA sponge.

Another aspect of the present disclosure provides a capturing agent or a capturing material that captures a microorganism, comprising a PVA sponge. In this aspect, the present disclosure also provides a method for capturing a microorganism or a part thereof, encompassing the step of contacting the microorganism or the part thereof with a PVA sponge.

Another aspect of the present disclosure provides a nucleic acid stabilizing agent or a nucleic acid stabilizing material, or a preserving agent or a preserving material that preserves a nucleic acid, comprising a PVA sponge. In this aspect, the present disclosure also provides a method for stabilizing or preserving a nucleic acid, encompassing the step of contacting a PVA sponge with the nucleic acid.

In one preferable embodiment, a PVA sponge may have an average pore size of about 80 μm to about 200 μm. In addition, or in an alternative embodiment, a PVA sponge may have a porosity of 89% to 91%.

In one embodiment, the present disclosure may also provide an agent for sampling or a material for sampling liquid biopsy, comprising the above-mentioned agent for suppression or material for suppression, or a kit comprising the above, and a composition therefor or a method therefor. The kit may be a kit for nucleic acid analysis (e.g., gene analysis), and the nucleic acid analysis may comprise the stem of nucleic acid amplification. Specifically, the kit, composition and method, and the like may be for analysis of a circulating nucleic acid (e.g., circulating cell-free nucleic acid).

As used herein, a “circulating nucleic acid” refers to a nucleic acid circulating in an organism (especially in blood). Among the circulating nucleic acids, a nucleic acid that does not comprise a cell is referred to as “circulating cell-free nucleic acid” herein. The circulating nucleic acid herein may be, for example, tumor circulating DNA or tumor circulating miRNA. As used herein, a “tumor circulating nucleic acid” refers to a circulating nucleic acid that is associated with or suspected to be associated with a tumor, wherein when the nucleic acid is a DNA, the nucleic acid is also called a “tumor circulating DNA”, and when the nucleic acid is an miRNA, the nucleic acid is also called a “tumor circulating miRNA”.

In one embodiment, the kit, composition and method, and the like of the present disclosure can be used for monitoring of the amount of tumor in a body, determination of an agent-resistant gene, early detection of cancer, or recurrence monitoring. In addition, or as an alternative, a circulating nucleic acid may be a cfDNA. In such a case, the kit, composition and method, and the like of the present disclosure may be used for prenatal diagnosis, detection of chromosomal abnormality, or neonatal screening. The kit, composition and method, and the like of the present disclosure may be used for detection of virus infection. In addition, the kit of the present disclosure may be used for absolute quantification of a nucleic acid. The absolute quantification may be carried out by a digital PCR.

The kit, composition and method, and the like of the present disclosure may be for capturing a circulating tumor cell. Such a kit, composition and method, and the like may be used for cancer prognosis prediction, or reexamination of a drug therapy method. The kit, composition and method, and the like of the present disclosure may be used to quantify tumor cell number using a digital PCR. Nucleic analysis may comprise a digital PCR.

As used herein, “digital PCR” refers to a method for detection and quantification of a nucleic acid, wherein the method is realized by directly counting the target molecule number without depending on the internal standard or endogenous control.

The kit, composition and method, and the like of the present disclosure may be used for absolute quantification of bacterium and virus in an oral cavity or in blood. The method of absolute quantification is known in the subject technical field, which can typically be realized by a method of preparing a calibration curve using a standard sample and measuring the absolute value of an unknown sample. A standard sample of which absolute amount (copy number) is already known and having the same sequence as an unknown sample is normally necessary. For example, it is possible to use, but not limited to, Bacteria (tufgene) Quantitative PCR Kit or the like that can be obtained from Takara Bio.

The kit, composition and method, and the like of the present disclosure can be used for preservation of a nucleic acid derived from blood. In addition, the kit, composition and method, and the like of the present disclosure may be used to separate plasma from blood for preservation.

In another aspect, the present disclosure may provide a sampling method for liquid biopsy, characterized by using a PVA sponge. The method may comprise the steps of:

the step of contacting the PVA sponge with the liquid biopsy;

the step of separating a nucleic acid specimen from the PVA sponge in water to obtain supernatant; and

the step of subjecting the supernatant to gene analysis.

The method may further comprise the step of drying the PVA sponge after the above-mentioned contacting step. In addition, the above-mentioned step of obtaining supernatant may comprise heating the PVA sponge in water and separating the nucleic acid specimen. The contact of the PVA sponge and the liquid biopsy may be carried out by immersing the PVA sponge in the liquid biopsy. Furthermore, a composition or kit for use in said method may also be provided.

The present disclosure may provide a concentration method for a sample. The method may comprise:

the step of contacting a PVA sponge with liquid biopsy;

the step of separating a nucleic acid specimen from the PVA sponge in water to obtain supernatant;

the step of freeze-drying the supernatant; and

the step of dissolving the freeze-dried supernatant in water.

The method may further comprise the step of drying the PVA sponge after the above-mentioned contacting step. In addition, the above-mentioned step of obtaining supernatant may comprise heating the PVA sponge in water and separating the nucleic acid specimen. The contact of the PVA sponge and the liquid biopsy may be carried out by immersing the PVA sponge in the liquid biopsy. Furthermore, a composition or kit for use in said method may also be provided.

In addition, the present disclosure provides a sampling method for adhered blood using a PVA sponge. The method may comprise:

the step of contacting the PVA sponge with dry adhered blood;

the step of separating a nucleic acid specimen from the PVA sponge in water to obtain supernatant; and

the step of subjecting the supernatant to gene analysis.

In addition, the above-mentioned step of obtaining supernatant may comprise heating the PVA sponge in water and separating the nucleic acid specimen. Furthermore, a composition or kit for use in said method may also be provided.

In addition, the present disclosure provides a method of quantitatively analyzing and capturing a microorganism. The method comprises:

the step of contacting a constant volume of PVA sponge with a specimen comprising a microorganism that is a target to be analyzed or captured;

the step of separating the microorganism or a component thereof from the constant volume of PVA sponge; and

the step of analyzing the microorganism or the component thereof as needed.

In addition, the above-mentioned separating step may comprise heating the PVA sponge in water and separating the microorganism or the component thereof. The component may be, for example, a nucleic acid in the microorganism. In addition, a composition or kit for use in said method may also be provided. Furthermore, the analysis to be carried out may be any analysis described in other parts herein. The analysis enables, for example, diagnosis of an infection, diagnosis of sepsis, and the like.

In another aspect, the present disclosure may provide an agent for sampling or a material for sampling comprising a PVA sponge, which is for analysis of a circulating nucleic acid in plasma or serum. In addition, the present disclosure may also provide a kit comprising said agent for sampling or material for sampling, which is for analysis of a circulating nucleic acid plasma or serum. The analysis of a circulating nucleic acid in plasma or serum may be quantitative analysis. The circulating nucleic acid may be a tumor circulating nucleic acid. The PVA sponge enables the analysis (e.g., quantitative reaction) of a nucleic acid in plasma or serum without freeing the nucleic acid by directly adding the PVA sponge to reaction liquid for PCR or the like after the addition of the plasma or serum. Not using the step of freeing a nucleic acid would more accurately reflect the total amount of the nucleic acid targeted for analysis, which may be very advantageous in quantification.

The present disclosure may provide a method of carrying out analysis of a circulating nucleic acid in plasma or serum, comprising: the step of contacting a PVA sponge with plasma or serum; and the step of subjecting the PVA sponge to gene analysis. In the method, the PVA sponge can be directly subjected to a reaction such as PCR. In addition, a composition or kit for use in said method may also be provided. Furthermore, the analysis to be carried out may be any analysis described in other parts herein. The analysis enables monitoring of a tumor, super early discovery, agent effect monitoring, and the like.

### General Technique

The molecular biological method, biochemical method and microbiological method used herein are methods that are well known and commonly used in the subject field, which are described in, for example, Sambrook J. et al. (1989). Molecular Cloning: A Laboratory Manual, Cold Spring Harbor and 3rd Ed. thereof (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press, Separate issue or Experimental Medicine, “Idenshi donyu & hatsugen kaiseki jikken hou (Gene introduction & expression analysis experimentation method)”, Yodosha, 1997, and the like, wherein the related portions (which may be the entirety) thereof are incorporated herein by reference.

DNA synthesis technique and nucleic acid chemistry for making an artificially synthesized gene are described in, for example, Gait, M. J.(1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and. Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press and the like, wherein the related portions thereof are incorporated herein by reference.

As used herein, “or” is used when it is possible to employ “at least one or more” of the matters listed in the sentence. When explicitly described, “within the range” of “two values” herein, the range includes the two values.

Reference literature such as scientific literature, patents, and patent applications cited herein is incorporated herein by reference to the same extent that the entirety of each document is specifically described.

The present disclosure has been explained while showing preferred embodiments to facilitate understanding. The present disclosure is explained hereinafter based on Examples. The above explanation and the following Examples are not provided to limit the present disclosure, but for the sole purpose of exemplification. Thus, the scope of the present disclosure is not limited to the embodiments and Examples that are specifically disclosed herein and is limited only by the scope of claims.

### EXAMPLES

### Example 1: Suppression of Amplification Inhibition by a PVA Sponge

The following experimentation demonstrates a PVA sponge's suppression of amplification inhibition by an inhibiting matter.

### Example 1A: Mouse Serum Human Genome DNA Addition/Collection Experimentation

<Experimentation>

A PVA sponge F(D) with a diameter of 4 mm and thickness of 3 mm or a water-soluble paper 120 MDP was immersed in 10 μL of mouse serum. After being naturally dried for about one hour, the sponge or water-soluble paper was added with 10⁵, 10⁴, 10³, 10² copies/10 μL of human genome DNAs to be naturally dried for a whole day and night. Each sample was added with 100 μL of DW and heated for 5 minutes at 95° C. After being heated, the sample was centrifuged for 5 minutes at 3000 rpm. 5 μL of each supernatant was added to a PCR master mix to carry out an amplification reaction.

(Adjustment of a Control Genome DNA)

Human Genomic DNA made by Promega was purchased and the copy number of the stock solution was calculated using a website (http://cels.uri.edu/gsc/cndna.html) to adjust a control solution with the copy number of 10⁵ to 10².

(Making a Mouse Serum-Added Control Sample)

A sponge with a diameter of 4 mm or a water-soluble paper was immersed in 10 mL of mouse serum, which was naturally dried for about one hour. The sponge or water-soluble paper immersed in the mouse serum is added with 10⁵ to 10² copies/10 μL of human genome DNA and naturally dried for a whole day and night.

(PCR Condition)

(1) When using THUNDERBIRD Probe qPCR Mix (TOYOBO)

TABLE 2
Composition of PCR master mix

| | |
|---|---|
| Reference Assay 20× (RNaseP) | 0.5 |
| 50× ROX | 0.2 |
| THUNDERBIRD* | 5 |
| Sample | 5 |
| | 10.7 |

(2) When using TaqPath qPCR Master Mix (Thermo Fisher Scientific Inc.)

TABLE 3
Composition of PCR master mix

| | |
|---|---|
| Reference Assay 20× (RNaseP) | 0.5 |
| TaqPath* | 5 |
| Sample | 5 |
| | 10.5 |

TABLE 4
Amplification condition
Program: 12K Standerd curve

| | | |
|---|---|---|
| 50° C., 2 min | | |
| 95° C., 10 min | | |
| 95° C., 15 sec | | |
| | {close oversize bracket} | 40 cycles |
| 60° C., 1 min | | |

<Result>

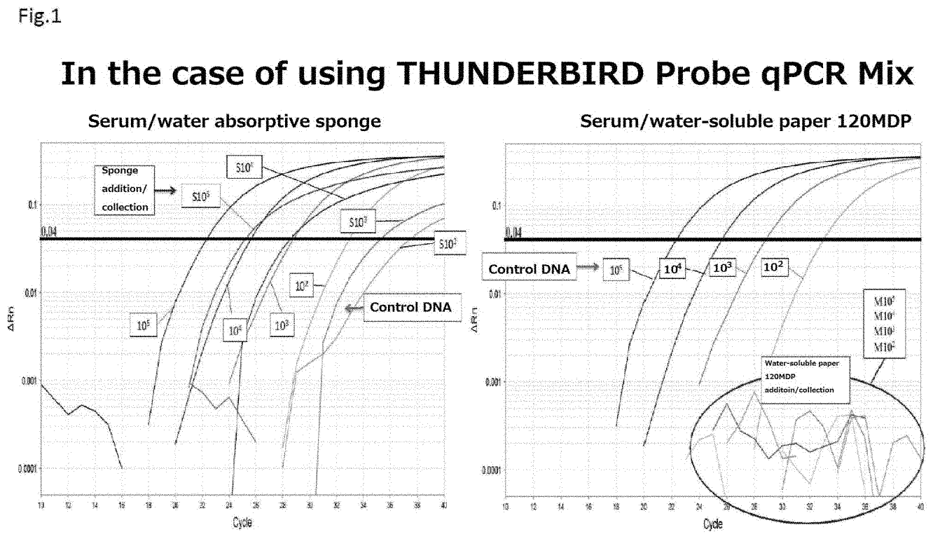

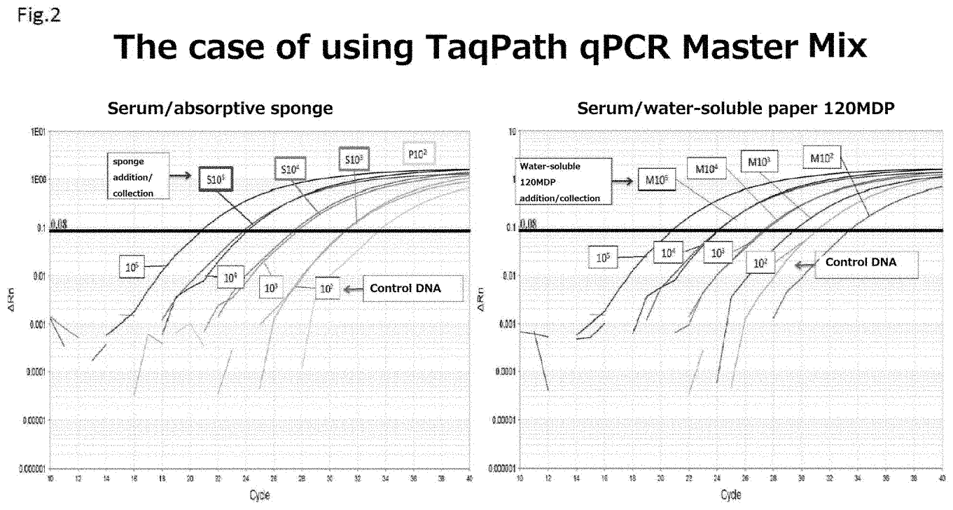

The result of amplification of each sample is shown in FIG. 1 and FIG. 2. When a PVA sponge is used, a human genome DNA added in serum is capable of amplification of RNase P gene together with THUNDERBIRD and TaqPath, but a water-soluble paper is capable of amplification of only TaqPath that is tolerant to gene amplification inhibiting matter.

<Observation>

The PVA sponge traps (captures) a PCR reaction inhibiting matter in serum and does not inhibit amplification of RNase P gene in a human genome DNA. It has been proven that it is possible to carry out gene amplification of a floating DNA in serum and detection using TaqMan Probe without carrying out the conventional method of extracting/purifying floating DNA in serum.

### Example 1B: Reaction Liquid Direct Addition of a PVA Sponge or Water-Soluble Paper Sample

<Experimentation>

After applying saliva comprising an oral mucosal cell gathered by swabbing or peripheral blood dropping to a PVA sponge F(D) with the thickness of 3 mm or water-soluble paper 120 MDP, drying was carried out for a whole day and night (about 16 hours). A sample in which the sponge or water-soluble paper was punched into a diameter of 2 mm was added to a PCR master mix to carry out RNase P gene amplification reaction in a human genome DNA.

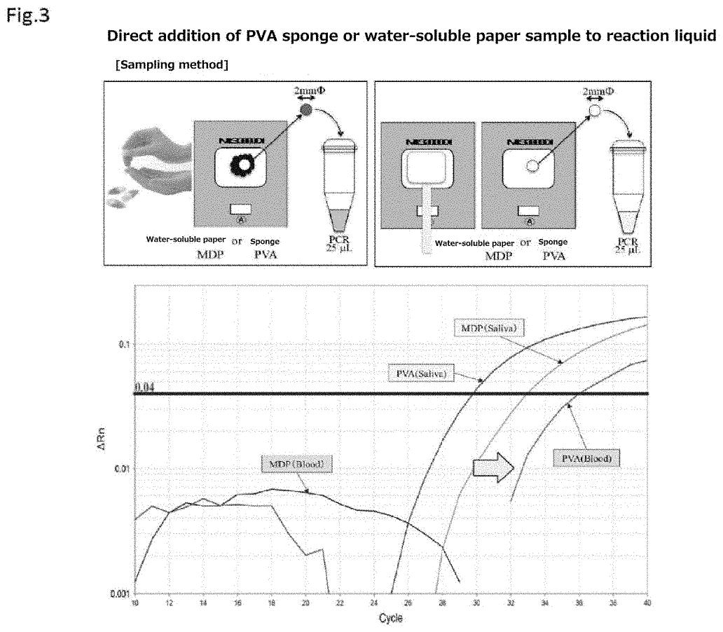

Sampling was carried out while using a disk in which the disk size of the PVA sponge F(D) or water-soluble paper 120 MDP is 2 mm Φ and in accordance with the sampling method shown in FIG. 3 for DNA capturing. The reaction liquid amount provided to the PCR was 25 μL.

TABLE 5
PCR reaction liquid composition (in the case of 25 μL)

| | |
|---|---|
| Reference Assay 20× (RNaseP) | 1.25 |
| 50× ROX | 0.5 |
| THUNDERBIRD | 12.5 |
| DW | 10.75 |
| | 25 |

TABLE 6
PCR condition

| | | |
|---|---|---|
| 95° C., 1 min | | |
| 95° C., 3 sec | | |
| | {close oversize bracket} | 40 cycles |
| 60° C., 30 sec | | |

<Result>

The amplification result is shown in FIG. 3. While RNase P gene amplification was observed together with blood/saliva with the PVA sponge F(D), in the case of the water-soluble paper MDP 120, gene amplification was carried out regarding saliva with less inhibition, but all amplification was not observed regarding blood.

<Observation>

The PVA sponge has greater effect of capturing PCR reaction inhibiting matter and suppressing amplification reaction regarding both blood and saliva compared to the water-soluble paper 120 MDP. Furthermore, even when adding a PVA sponge piece (2 mm ϕ and thickness of 3 mm) to a reaction liquid, there is no observation of light path inhibition of a real-time PCR apparatus system detecting fluorescence emitted from a TaqMan probe and amplification reaction inhibition. The PVA sponge piece was directly added to the PCR reaction liquid to suggest the possibility of amplification reaction.

### Example 1C: Reaction Inhibition Test of a Serum-Treated PVA Sponge

<Experimentation>

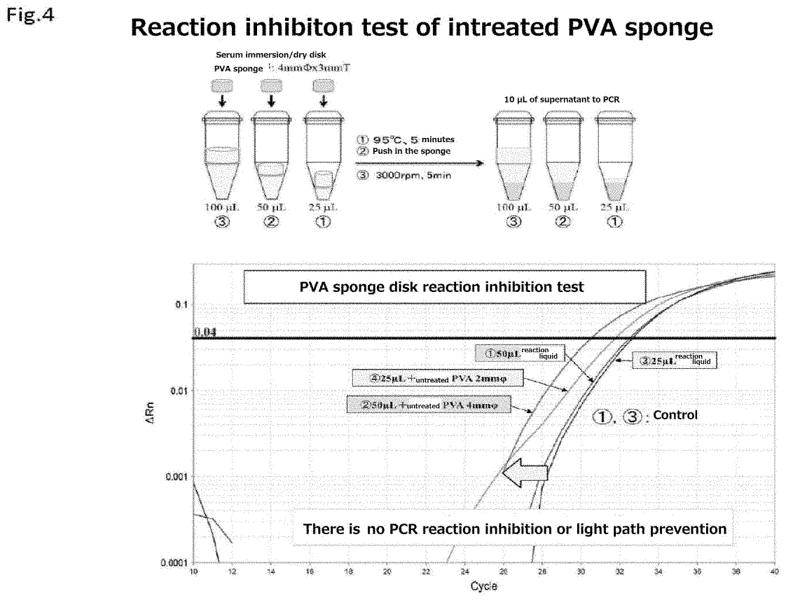

An experimentation regarding the presence/absence of amplification reaction inhibition and measurement-system light path prevention is the case of directly adding an untreated PVA sponge F(D) to a PCR reaction liquid. A Master Mix in which genome DNA 5.0×10⁴copy is added to a PCR reaction liquid is adjusted, wherein a PVA sponge with the diameter of 2 mm×thickness of 3 mm is added to 25 μL and a PVA sponge with the diameter of 4 mm×thickness of 3 mm is added to a 50 μL system to carry out RNase P gene amplification reaction. The process of the sampling is shown in FIG. 4.

TABLE 7
PCR reaction liquid composition (in the case of 50 μL)

| | |
|---|---|
| Reference Assay 20× (RNaseP) | 2.5 |
| 50× ROX | 1.0 |
| THUNDERBIRD | 25 |
| DW | 16.5 |
| Genome DNA(1.0 × 10⁴ copy/μL) | 5 |
| | 50 |

TABLE 8
Reaction condition

| | | |
|---|---|---|
| 95° C., 1 min | | |
| 95° C., 3 sec | | |
| | {close oversize bracket} | 40 cycles |
| 60° C., 30 sec | | |

<Result>

The result is shown in FIG. 4. The tendency of the Ct value being the same level or lower has been observed in sponge addition compared to additive-free (1) and (3). When comparing the Ct (Threshold Cycle) value of an amplification curve with the PVA sponge additive-free control, the presence of a PVA sponge F(D) in a reaction liquid does not inhibit PCR reaction and light path.

<Observation>

Even when a PVA sponge piece (4 mm or 2 mmϕ and the thickness of 3 mm) is added to a reaction liquid, there is no observation of light path inhibition of a real-time PCR apparatus system detecting fluorescence emitted from a TaqMan probe and amplification reaction inhibition. The PVA sponge piece was directly added to the PCR reaction liquid to suggest the possibility of amplification reaction.

### Example 1D: Reaction Inhibition Test of a Serum-Treated PVA Sponge

<Experimentation>

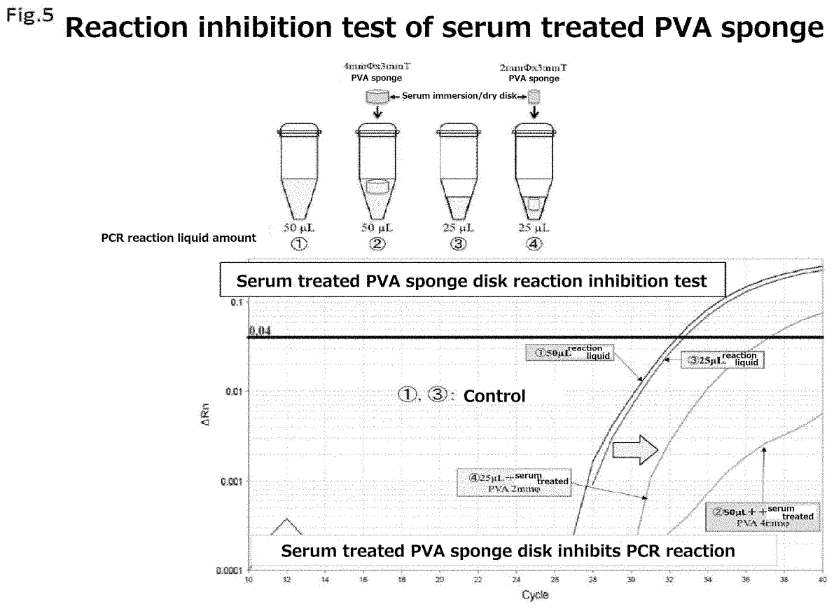

An experimentation regarding the presence/absence of amplification reaction inhibition and measurement-system light path prevention in the case of directly adding a serum-treated PVA sponge F(D) to a PCR reaction liquid. A Master Mix in which genome DNA 5.0×10⁴ copy is added to a PCR reaction liquid is adjusted, wherein a serum-treated PVA sponge with the diameter of 2 mm×thickness of 3 mm is added to 25 μL and a serum-treated PVA sponge with the diameter of 4 mm×thickness of 3 mm is added to a 50 μL system to carry out RNase P gene amplification reaction. The process of the sampling is shown in FIG. 5.

The condition of the sampling is described below.

### Time: 0, 1, 2, 4 hr

PVA: 4 mmΦ or 2 mmΦ disk
Method: Immerse PVA in serum
Immersion liquid amount: about 25 μL or about 5 μL
Drying: about 16 hours until the next morning

TABLE 9
PCR reaction liquid composition (in the case of 50 μL)

| | |
|---|---|
| Reference Assay 20× (RNaseP) | 2.5 |
| 50× ROX | 1.0 |
| THUNDERBIRD | 25 |
| DW | 16.5 |
| Genome DNA(1.0 × 10⁴ copy/μL) | 5 |
| | 50 |

TABLE 10
Reaction condition

| | | |
|---|---|---|
| 95° C., 1 min | | |
| 95° C., 3 sec | | |
| | {close oversize bracket} | 40 cycles |
| 60° C., 30 sec | | |

The result is shown in FIG. 5. The Ct value is greater compared to those that are additive-free and inhibition was recognized in serum-treated sponge (2) and (4). However, the inhibition level decreased when the disk size is made smaller (small amount of serum). When comparing the Ct (Threshold Cycle) value of amplification curve with that of a PVA sponge additive-free control, gene amplification reaction inhibition of PCR is observed from the presence of a serum-treated PVA sponge F(D) in a reaction liquid.

<Observation>

While amplification reaction inhibition was observed when a serum-treated PVA sponge piece (4 mm or 2 mmϕ and the thickness of 3 mm) is added to a reaction liquid, the possibility of optimization of this reaction was shown by examining the reaction liquid amount, PVA sponge size and the serum pre-treatment process.

### Examples 1-1: Serum Gathering Device 1

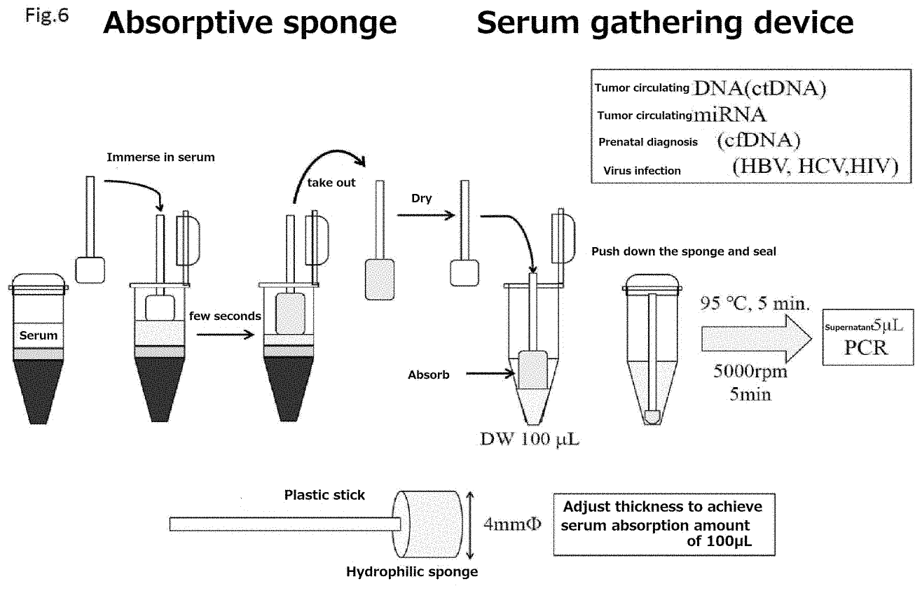

One example of the embodiment of the present disclosure is shown in FIG. 6. A device wherein a hydrophilic sponge (e.g., PVA sponge) is turned into a cylindrical sponge (e.g., 4 mmϕ) with a constant diameter and a plastic stick is attached as a handle is provided. It is possible to adjust the thickness of the sponge and adjust the liquid amount retained by a sponge to a constant amount such as 100 μL.

Blood is separated in an Eppendorf tube and the sponge portion of the device is immersed in the serum portion for a few seconds. Then, the device is taken out for drying. After the drying, the sponge is immersed in distilled water. For example, the action can be carried out in an Eppendorf tube. The sponge is pushed down for sealing, heat-treated for 5 minutes at 95° C. and centrifuged for 5 minutes at 5000 rpm. Then, 5 μL of supernatant is provided to the PCR.

A sample is provided in such a manner to enable detection of tumor circulating DNA (ctDNA), detection of tumor circulating miRNA, detection of cfDNA (prenatal diagnosis), detection of virus infection (HBV, HCV, HIV and the like) and the like.

### Examples 1-2: Serum Gathering Device 2

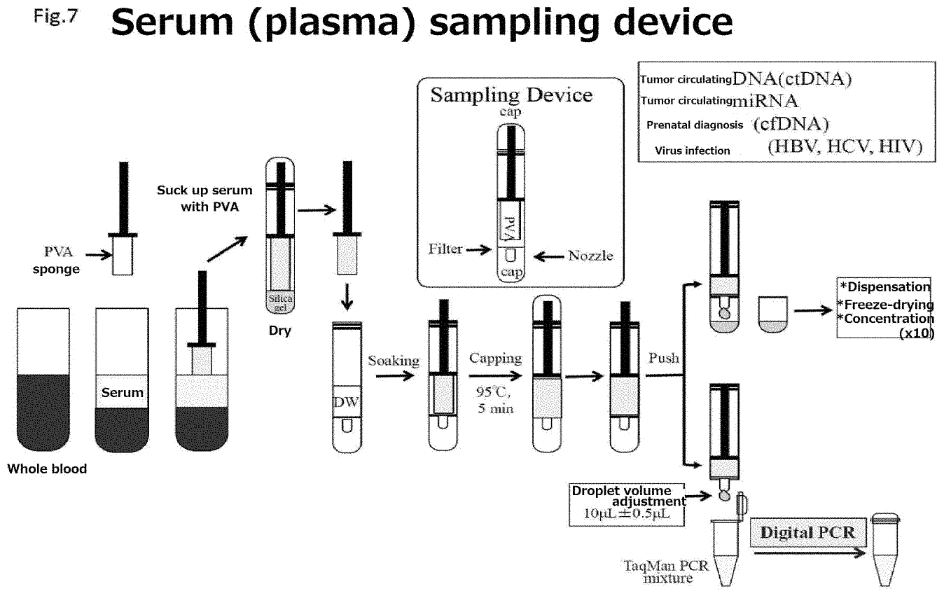

The example of the preferable embodiment of the present disclosure is shown in FIG. 7. A serum (or plasma) sampling device comprises a jig comprising a PVA sponge and a tube-like body storing the jig, wherein the body comprises a cap, filter and a nozzle. A PVA sponge is immersed in whole blood or serum to be soaked up with the PCA. Then, the PVA is dried. The PVA can be naturally dried, but the PVA can also be dried using silica gel. The dried PVA is immersed in distilled water in the device and the cap is closed to be heated for 5 minutes at 95° C. Then, by pushing the jig, the supernatant of the distilled water is pushed out from the nozzle through a filter. The supernatant can be dispensed, freeze-dried, concentrated, or the like. In addition, the volume of the pushed out supernatant can be adjusted to be constant such as 10 μL±0.5 μL. This constant volume of supernatant can be put in a PCR reaction liquid and provided to a digital PCR or the like.

This can enable detection of tumor circulating DNA (ctDNA), detection of tumor circulating miRNA, detection of cfDNA (prenatal diagnosis), detection of virus infection (HBV, HCV, HIV and the like) and the like.

### Example 2: Capturing of a Cell by a PVA Sponge

(Example 2A: Stability of White Blood Cell in a PVA Disk)

<Experimentation>

A sample in which 10 μL peripheral blood gathered from a fingertip using a lancet is diluted with 10 μL of PBS+1mMEDTA was dropped on a PVA 4 mmϕ disk for incubation (0, 1, 2, 4, 8 hours) in a sealed container (Eppendorf tube) at room temperature. After the PVA disk after each time has passed has been dried for one night at room temperature, the disk was treated in the following procedure.

5 μL of supernatant obtained by centrifugation for 3 minutes at 3000 rpm after immersion in 50 μl of distilled water and heating for 5 minutes at 95° C. is added to a PCR master mix to perform RNase P gene amplification reaction.

<Result>

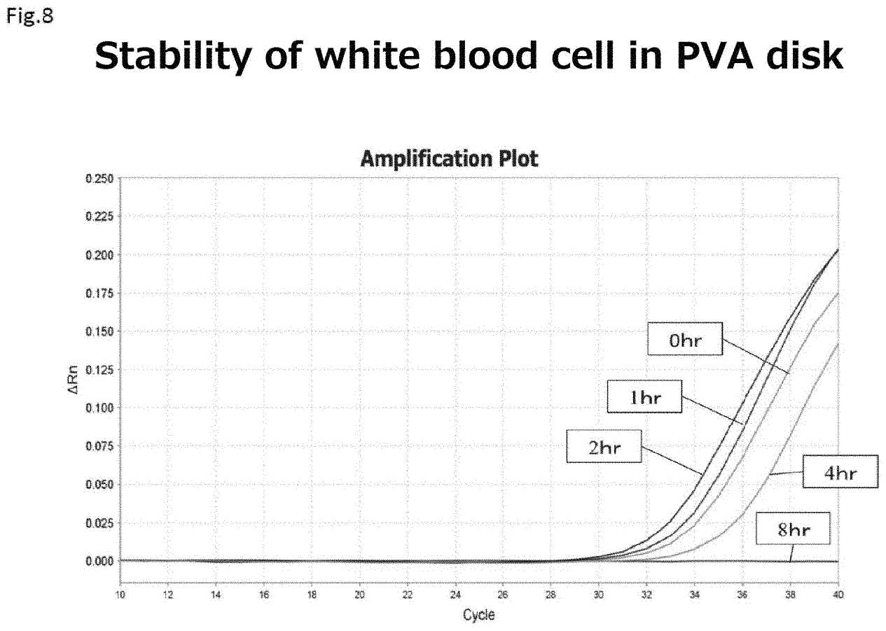

The result is shown in FIG. 8. From the Ct (Threshold Cycle) value of the amplification curve, the gene amplification (ΔCt: about 1 cycle) is substantially equivalent with incubation within 0 to 2 hours, wherein the amplification is ΔCt: about 3 cycles delayed in four hours. In 8 hours, amplification was not at all observed.

<Observation>

It is considered that adherence of white blood cells in whole blood to the PVA sponge would be maximum in two hours, and then the white blood cells would be dissolved and be lost in 8 hours. The peripheral blood immersed in the PVA sponge desirably enters into the drying step within two hours, and it has been demonstrated that the process of entering into the drying step quickly after the immersion is preferred.

(Example 2B: Selection of a PVA Sponge)

<Experimentation>

(Sample Adjustment)

PVA sponges D(D), E(D), F(D) and Y(D) were used to drop 5 μL of peripheral blood to a 4 mmϕ Disk of each PVA to be dried for one night.

(PVA Sponge PBS Washing)

Each dried PVA sponge disk is immersed in 200 μL PBS and the disk is taken out after vortex. After heating the PBS solution of the supernatant for 5 minutes at 95° C., 1 μL of the supernatant that has been centrifuged (5 minutes at 3000 rpm) was diluted with 4 μL of distilled water and added to a PCR master mix to perform RNase P gene amplification reaction.

(PBS Washing PVA Sponge Disk Treatment)

5 μL of supernatant that has been heated for 5 minutes at 95° C. and centrifuged (3000 rpm, 5 minutes) after immersing and vortex of each disk in 200 μL of DW is added to a PCR master mix to perform RNase P gene amplification reaction.

<Result>

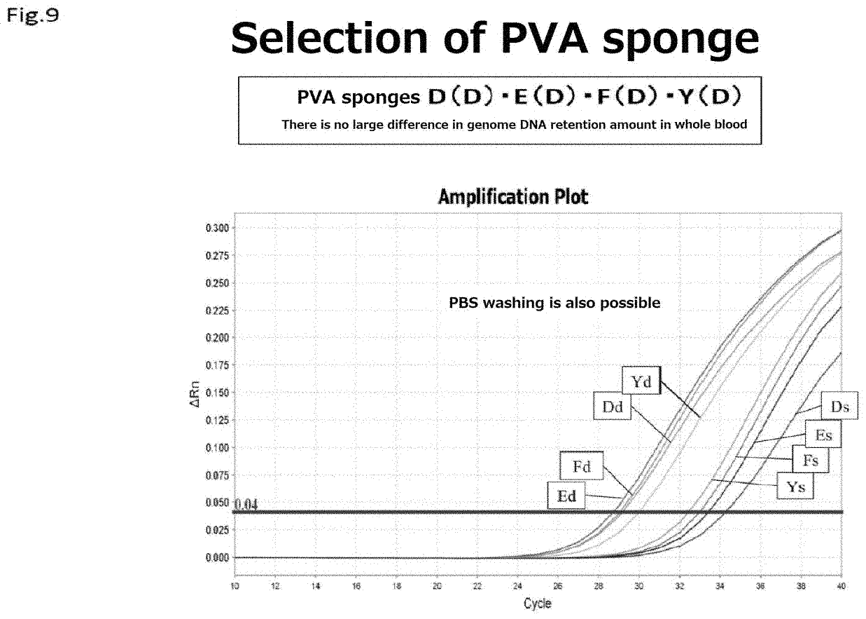

The result is shown in FIG. 9.

PVA sponge PBS wash supernatant PCR gene amplification: Ds, Es, Fs, Ys
PBS washing PVA sponge disk treated supernatant PCR gene amplification: Dd, Ed, Ed, Yd

Regarding the result of the gene amplification, there was no difference based on the type of the PVA sponge. It is understood that the majority of white blood cells are adhered to a PVA sponge than freed white blood cells in a PBS washing liquid from the Ct value.

<Observation>

The PVA sponge has been proven to have the performance of adhering cells.

(Example 2C: PVA Disk Whole Blood Retaining Amount)

<Experimentation>

1, 5, 10, 20 μL of peripheral blood was dropped to a PVA sponge (F(D) 4 mmϕ×3 mmT) disk and dried for one night. Then, the disk was treated in the following procedure.

5 μL of supernatant that has been centrifuged (3000 rpm, minutes) after each dried PVA sponge disk has been immersed in 200 μL distilled water and heated for 5 minutes at 95° C. was added to a PCR master mix to perform RNase P gene amplification reaction.

<Result>

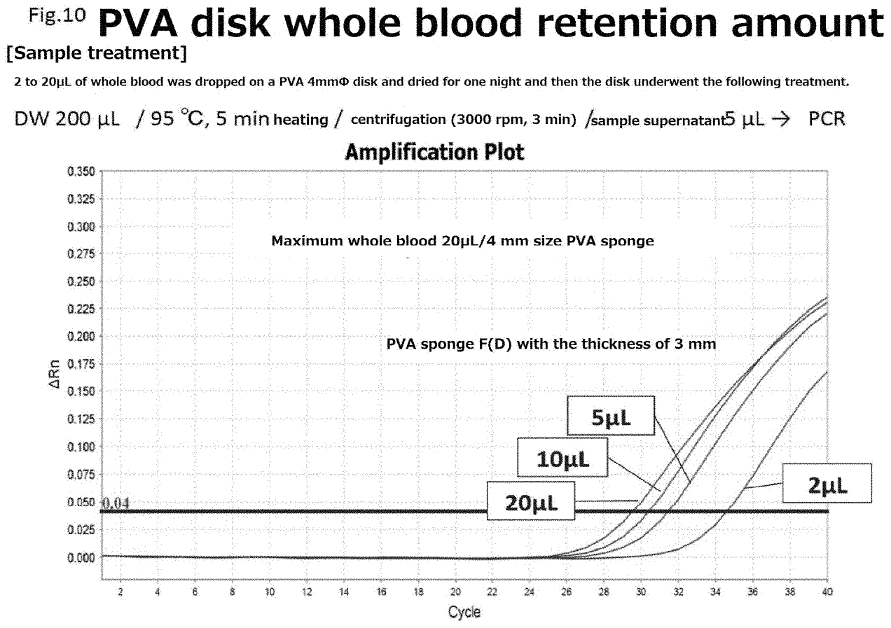

The result is shown in FIG. 10. The Ct value of a quantitative amplification curve has been observed depending on the peripheral blood amount that has been dropped on the PVA sponge.

<Observation>

It is presumed that the maximum blood absorption amount of the PVA sponge (F(D) 4 mmϕ×3 mmT) is up to 25 μL and there is no PCR gene amplification reaction inhibition by the blood composition. Therefore, the appropriateness as a device that absorbs the liquid sample (liquid biopsy) up to the maximum absorption amount of a PVA sponge and detects the DNA in the sample has been proven.

(Example 2D: Capturing of White Blood Cell/Peeled Epithelial Cell in Saliva using a PVA Sponge)

<Experimentation>

Saliva secreting near a salivary gland in an oral cavity is gathered in a Eppendorf tube using a dropper a four PVAF(D) 2 mmϕ×3 mmT disks were immersed in a saliva sample. Incubation (each 0, 0.5, 1 and 2 hours) was carried out at room temperature. After passing each time, the PVA disk is dried for one night at room temperature and then the disk was treated in the following procedure. The dried PVA sponge was directly added to a PCR master mix to perform RNase P gene amplification reaction. The saliva immersion amount of PVA sponge of 2 mmϕ is about 5 μL, and the cell composition in the saliva is white blood cell: 10² to 10⁴ cells/μL and peeled epithelial cell: 10² to 10⁴ cells/μL. Thus, it is considered that the captured amount is about 10⁵ cells/PVA disk.

TABLE 11
PCR reaction liquid composition (in the case of 25 μL)

| | |
|---|---|
| Reference Assay 20× (RNaseP) | 1.25 |
| 50× ROX | 0.5 |
| THUNDERBIRD | 12.5 |
| DW | 10.75 |
| | 25 |

TABLE 12

| | | |
|---|---|---|
| 95° C., 1 min | | |
| 95° C., 3 sec | | |
| | {close oversize bracket} | 50 cycles |
| 60° C., 30 sec | | |

<Result>

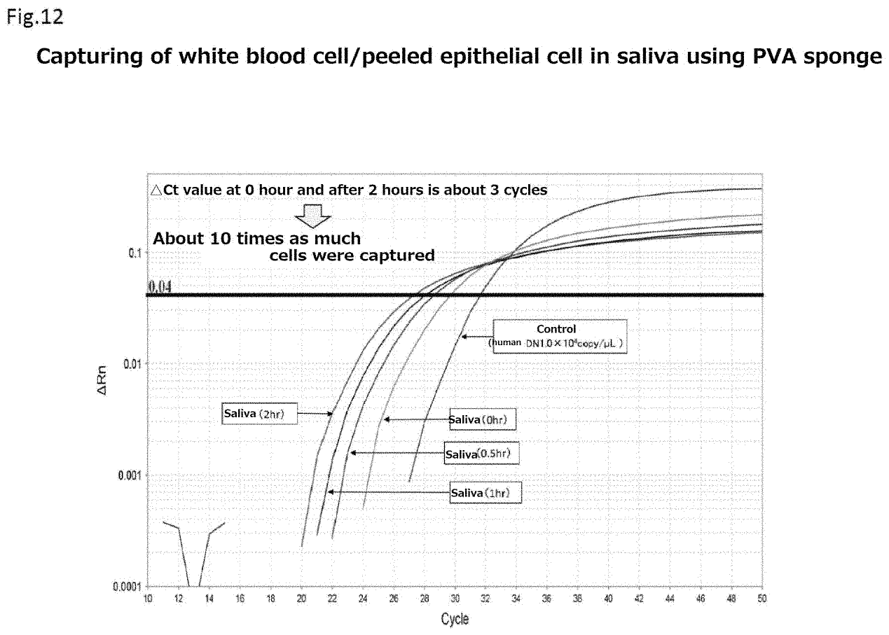

The result is shown in FIG. 12. From the Ct (Threshold Cycle) value of the amplification curve, the Ct value of the amplification curve became smaller with the incubation within 0 to 2 hours. The captured cell number is doubled from the result, 0 hour: 30 cycles, 0.5 hour: 29 cycles, 1 hour: 28 cycles, and 2 hours: 27 cycles.

<Observation>

Since the captured cell number is increased over time, the white blood cell/peeled epithelial cell is presumed to be adhered to a PVA. It has been proven that a PVA sponge has cell adhering ability also in saliva. Although not to be bound by any theory, it can also be considered that the PVA is a scaffold of a cell.

The present method is suitable for carrying out gene analysis of a reproductive cell line that does not change for lifetime by holding a PVA sponge in an oral cavity and subjecting the PVA sponge to gene analysis after drying the PVA sponge.

Even saliva that is found out to inhibit amplification reaction of polymerase in the same manner as blood was discovered to have the suppressing effect by a PVA sponge in the same manner.

(Example 2E: Direct Sampling in an Oral Cavity using a PVA Sponge)

<Experimentation>

8 volunteers held a PVA sponge disk (F(D) 2 mmϕ×3 mmT) near a salivary gland under a tongue for about one minute, wherein the PVA sponge disk was dried for one night after being taken out. The dried PVA sponge disk is directly added to a PCR master mix to perform RNase P gene amplification reaction.

TABLE 13
PCR reaction liquid composition (in the case of 25 μL)

| | |
|---|---|
| Reference Assay 20× (RNaseP) | 1.25 |
| 50× ROX | 0.5 |
| THUNDERBIRD | 12.5 |
| DW | 10.75 |
| | 25 |

TABLE 14

| | | |
|---|---|---|
| 95° C., 1 min | | |
| 95° C., 3 sec | | |
| | {close oversize bracket} | 50 cycles |
| 60° C., 30 sec | | |

<Result>

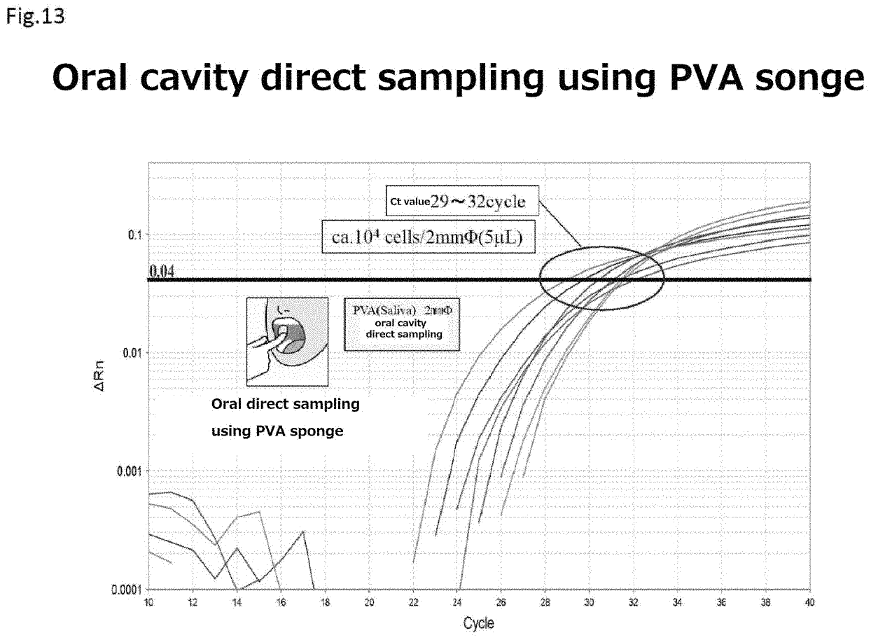

The result is shown in FIG. 13. The Ct (Threshold cycle) values of the amplification curve of 8 people were observed in 29 to 32 cycles, and from the comparison with the control genome DNA, the cell number that was able to be captured with a PVA sponge from white blood cell/peeled epithelial cell in saliva was about 10⁴ cells. In the case of whole blood, the difference between a 2 mmϕ PVA sponge in oral cavity and a water-soluble paper (120 MPD) was minor.

<Observation>

The present method demonstrated that it is possible to prepare a sample suitable for gene analysis of a reproductive cell line that does not change for lifetime by a simple sampling in which a PVA sponge is held in an oral cavity and the PVA sponge is dried.

In addition, while the cell mount in saliva generally greatly differs depending on the individual, as discussed above, the Ct values of the amplification of curve of 8 people were within the constant range. Although not wishing to be bound by any theory, it can be considered that the PVA sponge was able to collect a constant amount of cells per volume of a sponge without depending on the cell amount in saliva by capturing a constant amount of cells in the cavity of the PVA sponge. Thus, such a property of a PVA sponge enables collection of a constant amount of microorganisms in a sample comprising microorganisms, whereby it is considered that comparison between samples is easy.

(Example 2-1: Saliva Sampling Process)

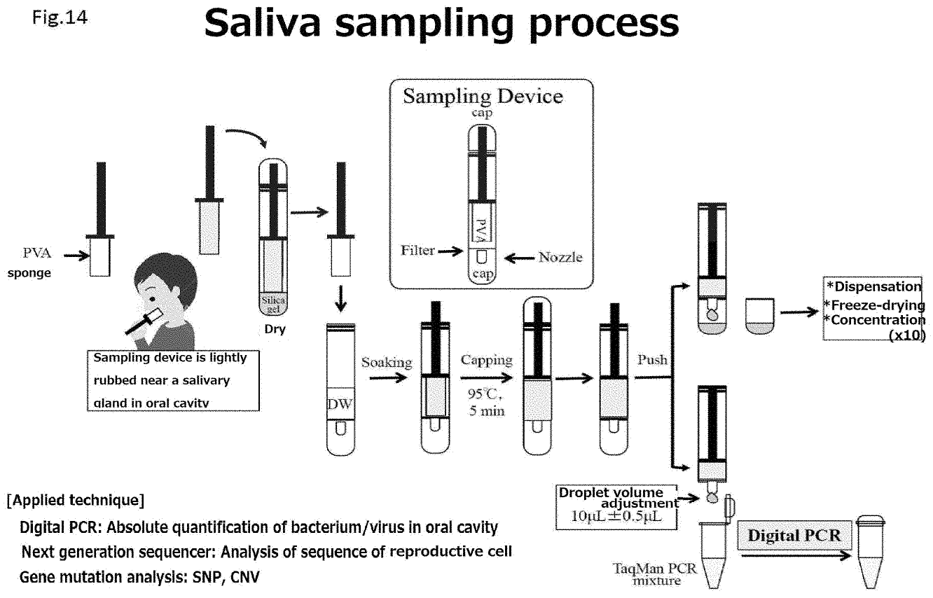

An example of a preferable embodiment of the present disclosure is shown in FIG. 14. A saliva sampling device comprises a jig comprising a PVA sponge and a tube-like body storing the jig, wherein the body comprises a cap, filter and a nozzle. The vicinity, of a salivary gland is lightly rubbed with a PVA sponge of a sampling device. The PVA sponge is dried using a silica gel. After the PVA sponge is dried, the PVA sponge is immersed in distilled water in a device, capped and heated for 5 minutes at 95° C. The jig is pushed down to push out supernatant of distilled water from the nozzle through the filter. The supernatant can be dispensed, freeze-dried, concentrated, or the like. In addition, the volume of the pushed out supernatant can be adjusted to be constant such as 10 μL±0.5 μL. This constant volume of supernatant can be put in a PCP reaction liquid and provided to a digital PCR or the like.

This enables absolute quantification of bacterium/virus in an oral cavity using a digital PCR, sequence analysis of a reproductive cell using a next generation sequencer, SNP by gene mutation analysis, detection of CNV, or the like.

(Example 2-2: Cancer Cell Sampling Process)

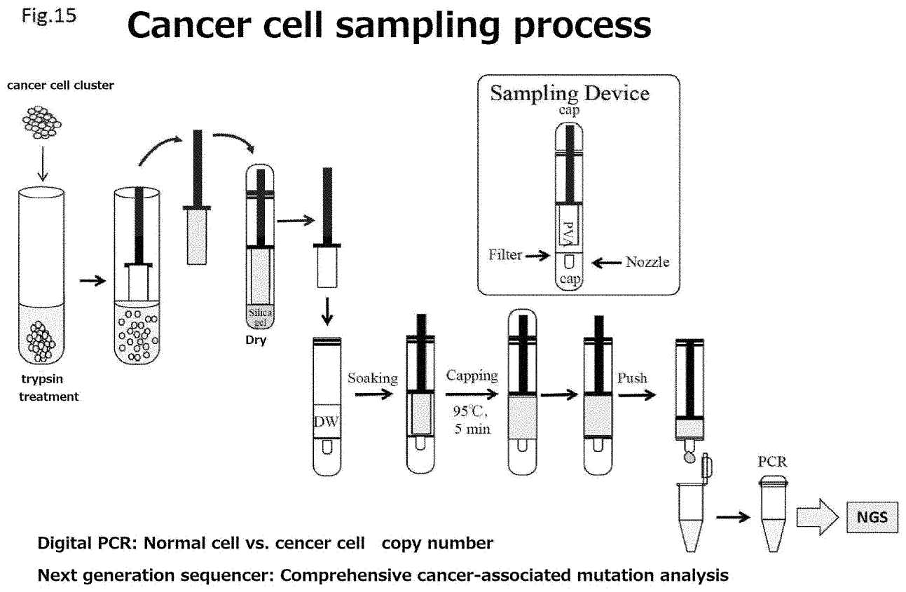

An example of a preferable embodiment of the present disclosure is shown in FIG. 15. A saliva sampling device comprises a jig comprising a PVA sponge and a tube-like body storing the jig, wherein the body comprises a cap, filter and nozzle.

Cancer cell cluster undergoes trypsin treatment. A PVA sponge is immersed in a reaction liquid that underwent trypsin treatment. The PVA sponge is dried using silica gel. After being dried, the PVA sponge is immersed in distilled water in a device, capped and heated for 5 minutes at 95° C. The jig is pushed down and the supernatant of the distilled water is pushed out from the nozzle through the filter. The supernatant is subjected to a reaction such as PCR.

This enables comparison of copy number between normal cells and cancer cells using a digital PCR and comprehensive cancer-related gene mutation analysis using a next generation sequencer.

(Example 2-3: Prompt Gene Examination of a Solid Tumor Cell)

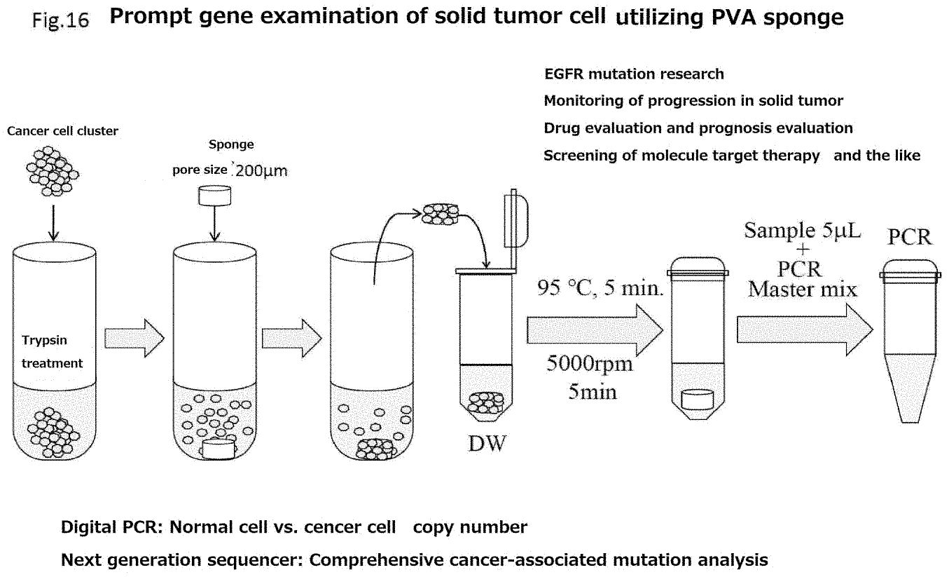

An example of a preferable embodiment of the present disclosure is shown in FIG. 16. A cancer cell cluster undergoes trypsin treatment. A PVA sponge is introduced to a reaction liquid that underwent trypsin treatment. For example, the sponge has a pore size of 200 μm. Then, the PVA sponge is taken out and immersed in distilled water. After being heated for 5 minutes at 95° C., supernatant is obtained by centrifugation for 3 minutes at 3000 rpm. The supernatant is subjected to gene analysis. For example, 5 μL of supernatant is mixed with a PCR master mix to be able to carry out PCR reaction or the like.

This enables comparison of copy number between normal cells and cancer cells using a digital PCR and comprehensive cancer-related gene mutation analysis using a next generation sequencer.

(Example 2-4: Agent Selectivity Test of a Solid Tumor Cell)

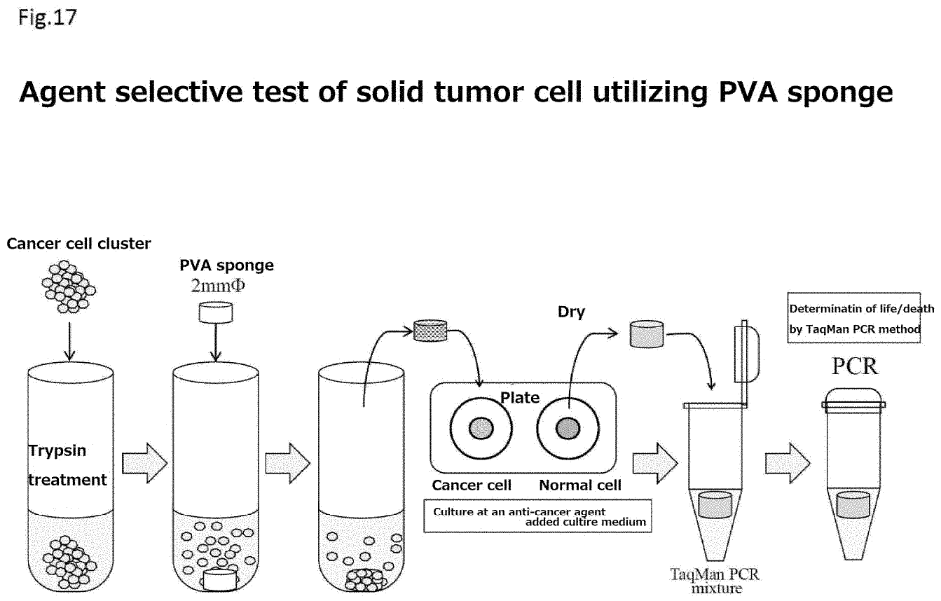

An example of a preferable embodiment of the present disclosure is shown in FIG. 17. A cancer cell cluster undergoes trypsin treatment. A PVA sponge is introduced to a reaction liquid that underwent trypsin treatment. For example, the PVA sponge is a 2 mmϕ disk. A disk attached with a cancer cell and a disk attached with a normal cell are cultured at an anti-cancer agent added culture medium. Then, the disk is dried and subjected to a PCR reaction. The life/death of a cell is determined with a Taqman PCR method to test the drug selectivity of a tumor cell.

### Example 3: DNA Stability Test

<Summary>

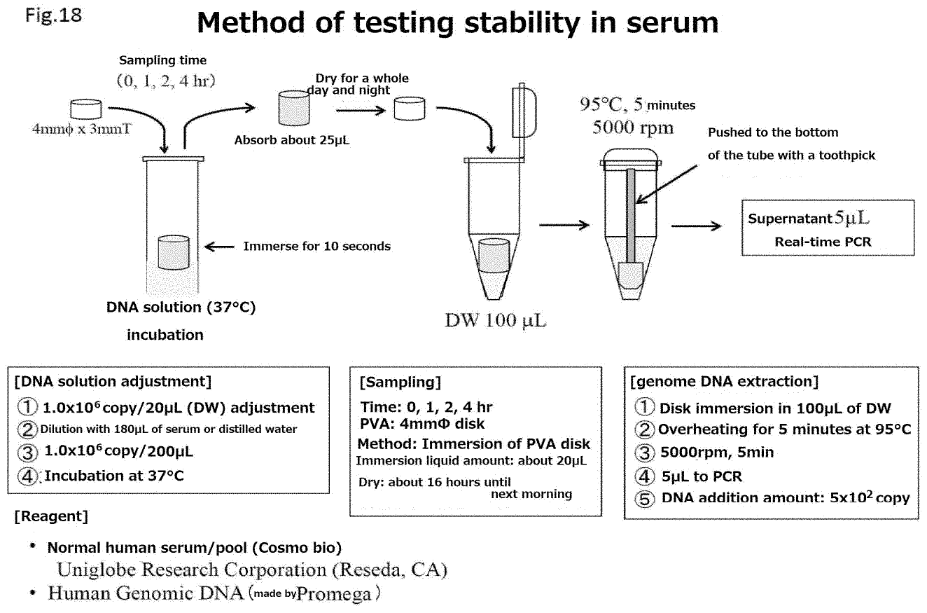

In the case of gene examination using liquid biopsy, cell death (necrosis (cytociasis) and apoptosis (active and functional cell death)) of white blood cell or peeled epithelial cell increases floating DNAs (cfDNAs), whereby it is common to make adjustment into serum (plasma) within 2 hours. In this regard, regarding the stability of free DNA in serum, genome DNA or plasmid DNA is added and a PVA sponge is used to examine the stability. The method of the Examination is also shown in FIG. 18.

<Material and Method>

(Preparation of Genome DNA or Plasmid DNA Serum Solution)

Genome DNA was used by preparing a commercially available (Human Genomic DNA: made by Promega) solution. Plasmid DNA was extracted/purified by inserting a gene fragment encoding an alcohol metabolism enzyme gene ADH1B to a plasmid derived from a commercially available retrovirus and culturing a genetic recombinant of Escherichia coli. Each was adjusted with the following procedure.

(1) 1.0×10⁶ copy/20 μL (DW) adjustment
(2) Dilution with commercially available serum (normal human serum/pool: Cosmo Bio) or 180 μL of distilled water
(3) 1.0×10⁶ copy/200 μL was prepared to perform stability test.

### (4) Incubation at 37° C.

(Sampling)

PVA sponge: 4 mmϕ disk

Method: A PVA disk is immersed in a solution adjusted with distilled water of serum or control

Immersion time: 0, 1, 2, 4 hr

Immersion liquid amount: about 20 μL

Drying time: about 16 hours until the next morning

(Genome DNA Extraction)

(1) Disk immersion in 100 μL of DW
(2) Heating for 5 minutes at 95° C.
(3) 5000 rpm, 5 min
(4) Put 5 μL in a PCR master mix
(5) The theoretical DNA addition amount added to a PCR reaction is calculated to be 5×10² copy

TABLE 15
PCR reaction liquid composition

| | |
|---|---|
| TaqMan Probe ADH1B 20× | 0.5 |
| 50× ROX | 0.2 |
| THUNDERBIRD | 5 |
| Template(plasmid DNA) | 5 |
| | 10.7 |

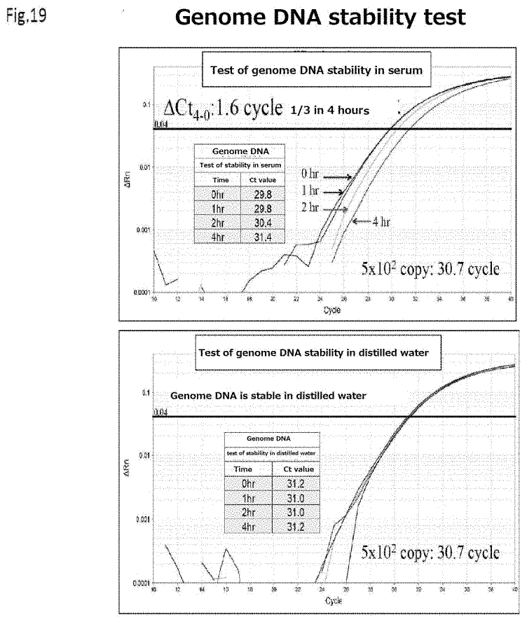

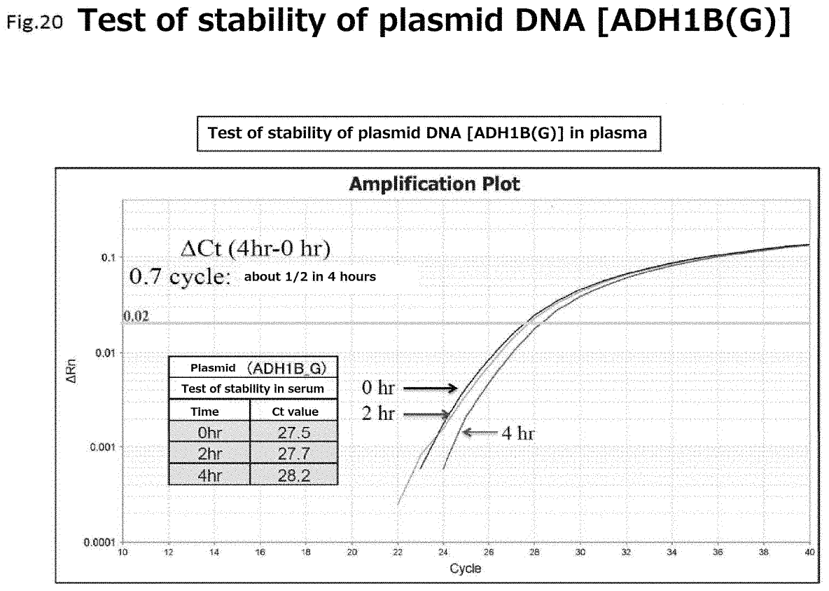

TABLE 16
Reaction condition

| | | |
|---|---|---|
| 95° C., 10 min | | |
| 95° C., 15 sec | | |
| | {close oversize bracket} | 40 cycles |
| 60° C., 1 min | | |

<Result>The result is shown in FIGS. 19 and 20. From the Ct (Threshold Cycle) value of an amplification curve, hardly any change of the Ct value of the amplification curve was observed with the incubation within 0 to 4 hours.

<Observation>

It has been proven that the genome DNA or plasmid DNA added in serum would stably exists within 4 hours. It has been proven that the free DNA (ctDNA) comprised after serum (plasma) separation is relatively stable. Stability was greater in plasmid DNA than genome DNA.

(Example 3-1: Plasma Separation/Preservation)

A device in which the portion of a filter (e.g., filter paper) in a filter plasma separation device is replaced with a PVA sponge is provided. Other configurations would be enough if they are in accordance with a commercially available plasma separation device (e.g., WATSON, FUKAEKASEI).

After whole blood is dropped on the filter portion and plasma component is separated, a piece was punched using a sample punch (Kai Corporation). The piece is added to a reaction liquid to enable use for gene amplification reaction such as PCR.

### Example 4: Freeze-Drying of Serum Treated PVA Sponge Adjustment Liquid

<Experimentation>

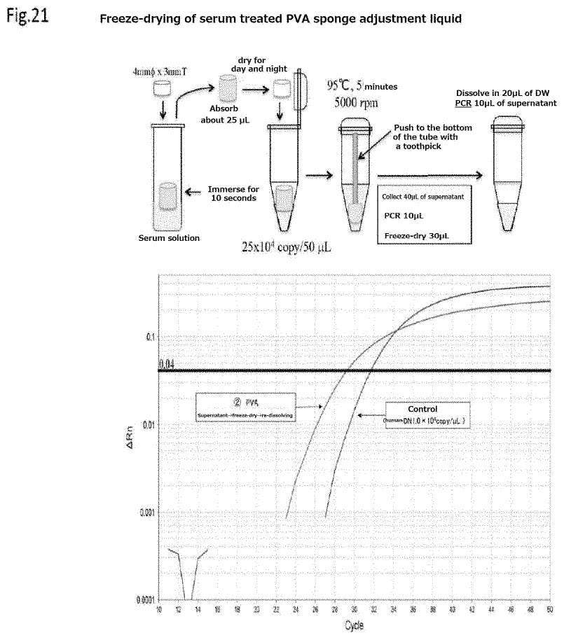

The procedure of the experimentation is shown in FIG. 21. More specifically, the experimentation was carried out in the following procedure.

(Experimentation Condition)

(1) Genome DNA: 25 μL of 1×10⁴ copy/μL solution is adjusted with DW×2(50 μL)
(2) A PVA sponge treated with 25 μL of serum is dried and immersed in the above-mentioned adjustment solution.
(3) After treatment for 5 minutes at 95° C., centrifugation at 3000 rpm
(4) Put 10 μL of supernatant to a PCR
(5) Freeze-drying about 30 μL of the remaining supernatant
(6) After freeze-drying, the residue is dissolved with 20 μL of DW
(7) 10 μL is used for PCR.

TABLE 17
PCR reaction liquid composition (in the case of 25 μL)

| | |
|---|---|
| Reference Assay 20×(RNaseP) | 1.25 |
| 50× ROX | 0.5 |
| THUNDERBIRD | 12.5 |
| DW | 10.75 |
| | 25 |

TABLE 18

| | | |
|---|---|---|
| 95° C., 1 min | | |
| 95° C., 3 sec | | |
| | {close oversize bracket} | 50 cycles |
| 60° C., 30 sec | | |

<Result>

The result is shown in FIG. 21, After being freeze-dried, the serum treated PVA sponge adjustment liquid caused amplification without a problem even when used for PCR as a re-dissolving sample.

<Observation>

It was suggested that, after being freeze-dried the supernatant of the serum treated PVA sponge adjustment liquid was dissolved with less DW, whereby enabling 2-fold to 10-fold concentration. Since PVA is a solid sponge, the supernatant can be easily dissolved. It is considered that a PCR reaction inhibition can easily be removed as a PVA sponge trap and precipitate.

### Example 5: Detection of Fiber/Paper/Table Dried Attached Blood

<Experimentation>

(Sample Treatment)

Blood attached to fiber/table is wiped off with a PVA 4 mmϕ disk comprising distilled water and treated with the following condition.

DW 200 μL/95° C., 5 min heating/centrifugation (3000 rpm, 3 min)/Sample supernatant 5 μL→PCR

<Result>

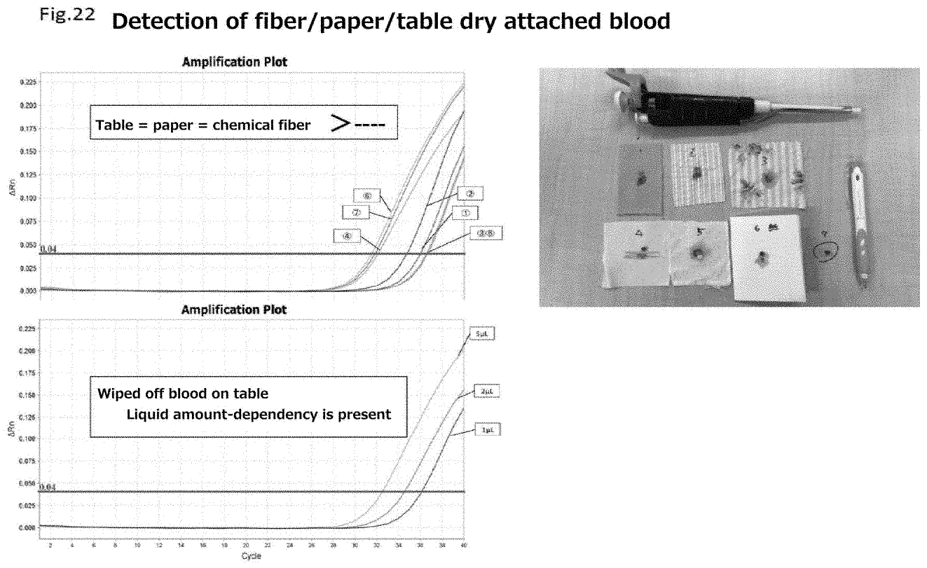

The result is shown in FIG. 22.

<Observation>

It is possible to utilize the function of a sponge of PVA to wipe off blood from clothes or table to which the blood is attached, dry the sponge, and then carry out gene examination. It is considered that the sponge can be used for crime investigation or the like.

### Example 6: System of Monitoring Cancer-Associated Gene in Blood

(Example 6A: Post-Surgery Early Recurrence Diagnosis)

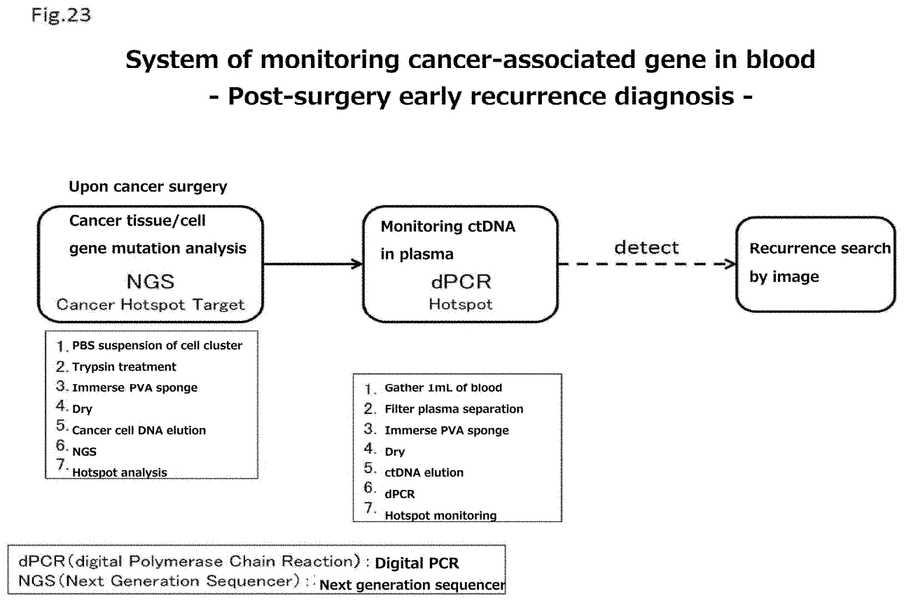

An example of post-surgery early recurrence diagnosis of the present disclosure is shown in FIG. 23.

Upon cancer surgery, cancer tissue/cell gene mutation analysis is carried out using a next generation sequencer (NGS) (e.g., available from Illumina, Thermo Fisher Scientific, Roche, or the like) in the following procedure. Specifically, Cancer Hotspot Target in which mutation is easily caused is targeted.

1. PBS suspension of a cell cluster
2. Trypsin treatment
3. Immersion of PVA sponge

### 4. Drying

5. Cancer cell DNA elution
6. Sequencing using NGS
7. Hotspot analysis

When a cancer-associated gene is detected, recurrence search using an image is carried out.

(Example 6B: Therapeutic Effect Monitoring)

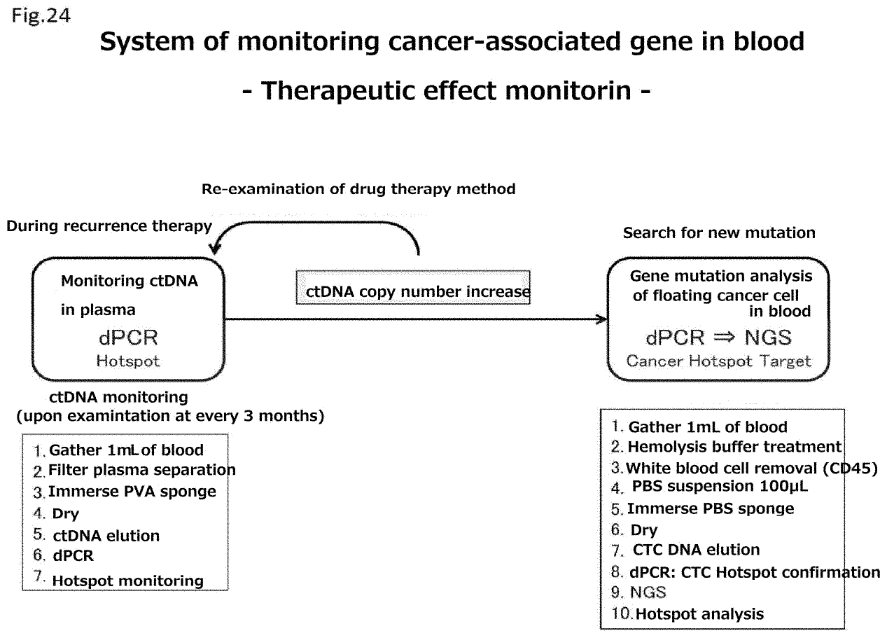

An example of a therapeutic effect monitoring using the present disclosure is shown in FIG. 24.

During recurrence therapy, ctDNA in plasma is monitored using dPCR in the following procedure. For example, monitoring is carried out upon examination performed every three months.

1. Blood collection (1 mL)
2. Filter plasma separation
3. Immersion of a PVA sponge

### 4. Drying

5. ctDNA elution
6. dPCR
7. Hotspot monitoring

When the copy number of the ctDNA is increased, re-examination of the drug therapy method is carried out.

As a search of a new mutation, gene mutation analysis of a cancer cell floating in blood is carried out using dPCR and NGS. Cancer Hotspot Target is targeted. CTC separation/purification is carried out in the following procedure.

1. Collect 1 mL of blood
2. Hemolysis buffer treatment
3. White blood cell removal (CD45)
4. 100 μL of PBS suspension
5. Immersing a PVA sponge

### 6. Drying

7. CTC DNA elution
8. dPCR: CTC Hotspot confirmation

### 9. NGS

10. Hotspot analysis

(Example 7A: PVA Sponge/Direct Quantification of cfDNA in Plasma)

<Experimentation>

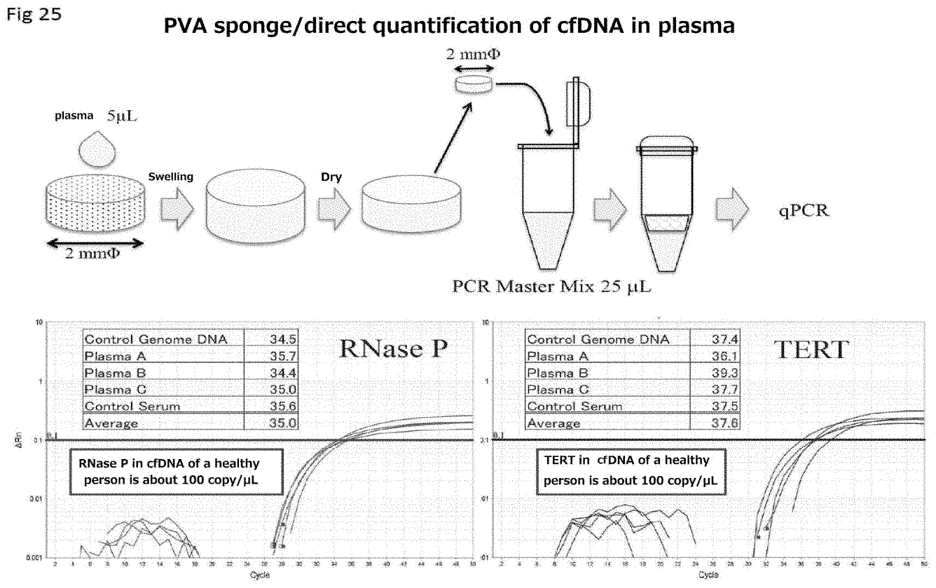

The procedure of the experimentation is shown in FIG. 25. More specifically, the experimentation was carried out in the following procedure.

(1) Plasma adjustment method: Supernatant in which the same amount of PBS+EDTA as whole blood is added and centrifugation is carried out for 10 minutes at 1500 G was used in the following experimentation.
(2) Genome DNA: 5 μL of 1×10² copy/μL (PBS+EDTA) solution is dropped/dried on a 2 mmϕ PVA sponge and then added to a PCR reaction liquid.
(3) 5 μL of each plasma is dropped/dried on a 2 mmϕ PVA sponge and then added to a PCR reaction liquid.

TABLE 19
PCR reaction liquid composition (in the case of 25 μL)

| | |
|---|---|
| Reference Assay 20× (RNaseP) | 1.25 |
| TaqPath | 12.5 |
| DW | 11.25 |
| | 25 |

TABLE 20

| | | |
|---|---|---|
| 95° C., 1 min | | |
| 95° C., 3 sec | | |
| | {close oversize bracket} | 50 cycles |
| 60° C., 30 sec | | |

<Result>

The result is shown in FIG. 25. A healthy person cfDNA was detectable in the CT value 35 to 37 cycles.

<Observation>

It is understood that a plasma added PVA sponge disk does not inhibit PCR reaction. In addition, from a PVA sponge added with plasma, amplification of a circulating nucleic acid was possible even when added to a reaction liquid without carrying out the step of freeing a nucleic acid. This suggests that the use of a PVA sponge enables quantification of circulating nucleic acid without extra purification steps. Less work in changing the entire volume such as elution is advantageous in accurate quantification.

(Example 7B: PVA Sponge/Direct Quantification of cfDNA in Plasma)

<Experimentation>

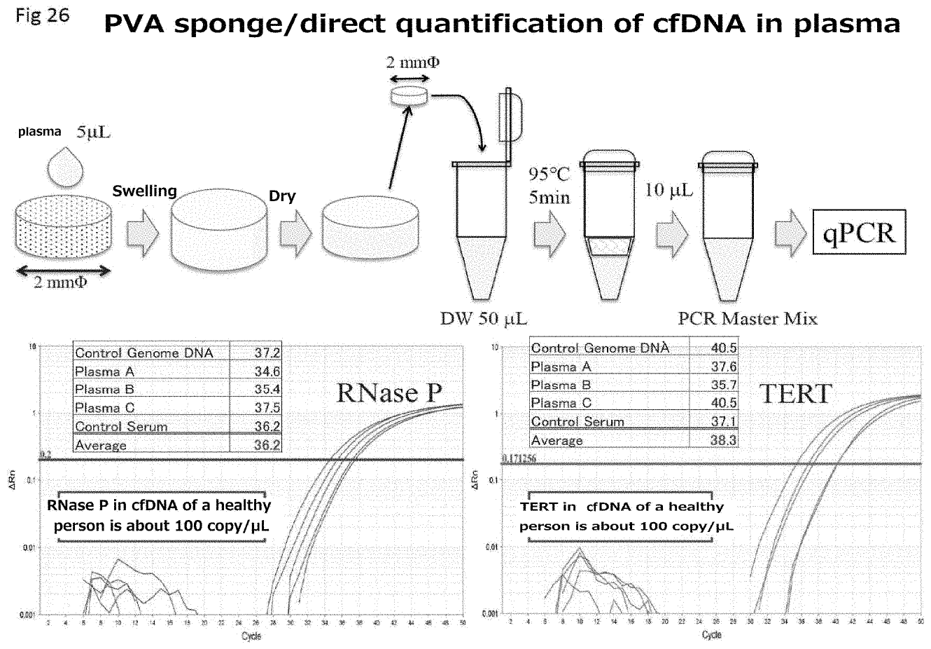

The procedure of the experimentation is shown in FIG. 26. More specifically, the experimentation was carried out in the following procedure.

(1) Plasma adjustment method: Supernatant in which the same amount of PBS+EDTA as whole blood is added and centrifugation is carried out for 10 minutes at 1500 G was used in the following experimentation.
(2) Genome DNA: 5 μL of 1×10² copy/μL (PBS+EDTA) solution. is dropped/dried on a 2 mmΦ PVA sponge, which was then treated for 5 minutes at 95° C. with 50 μL of DW.
(3) 10 μL is added to a PCR Master Mix to perform PCR.

TABLE 21
PCR reaction liquid composition (in the case of 25 μL)

| | |
|---|---|
| Reference Assay 20× (RNaseP) | 1.25 |
| TaqPath | 12.5 |
| Sample | 10.0 |
| DW | 1.25 |
| | 25 |

TABLE 22

| | | |
|---|---|---|
| 95° C., 1 min | | |
| 95° C., 3 sec | | |
| | {close oversize bracket} | 50 cycles |
| 60° C., 30 sec | | |

<Result>

The result is shown in FIG. 26. A healthy person cfDNA was detectable in the CT value 35 to 40 cycles.

<Observation>

It is understood that plasma added PVA sponge disk does not inhibit PCR reaction. It is shown that the use of a PVA sponge enables quantitative analysis of cell-free DNA in plasma.

(Note)

As disclosed above, the present disclosure is exemplified by the use of its preferred embodiments. However, it is understood that the scope of the present disclosure should be interpreted solely based on the Claims. It is also understood that any patent, any patent application, and any references cited herein should be incorporated herein by reference in the same manner as the contents are specifically described herein. The present application claims priority to Japanese Patent Application 2019-4438 (filed on Jan. 15, 2019) and Japanese Patent Application 2019-68152 (filed on Mar. 29, 2019). The entire content thereof is incorporated herein by reference.

### INDUSTRIAL APPLICABILITY

The present disclosure can be used for sample preparation in research or clinical purposes, for example, sample preparation of genotype, or sample preparation for pharmacokinetics monitoring.

1. An agent for suppression or a material for suppression that suppresses polymerase amplification activity inhibition effect by body fluid or a component thereof, comprising a PVA sponge.

2. The agent for suppression or the material for suppression of claim 1, wherein the PVA sponge has an average pore size of 80 μm to 200 μm.

3. The agent for suppression or the material for suppression of claim 1, wherein the PVA sponge has a porosity of 89% to 91%.

4. The agent for suppression or the material for suppression of claim 1, wherein the body fluid is blood or saliva.

5. An agent for sampling or a material for sampling a liquid biopsy, comprising the agent for suppression or the material for suppression of claim 1.

6. A kit comprising the agent for sampling or the material for sampling according to claim 5.

7. The kit of claim 6, which is for nucleic acid analysis.

8. The kit of claim 7, wherein the nucleic acid analysis comprises the step of amplification of a nucleic acid.

9. The kit of claim 7, which is for analysis of a circulating nucleic acid.

10. The kit of claim 9, wherein the circulating nucleic acid is a tumor circulating DNA or a tumor circulating miRNA.

11. The kit of claim 10, which is for monitory of tumor amount in a body, determination of agent resistant gene, early detection of cancer, or recurrence monitoring.

12. The kit of claim 9, wherein the circulating nucleic acid is cfDNA.

13. The kit of claim 12, which is for prenatal diagnosis.

14. The kit of claim 12, which is for detection of chromosomal abnormality or neonatal screening.

15. The kit of claim 9, which is for detection of virus infection.

16. The kit of claim 7, which is for absolute quantification of a nucleic acid.

17. The kit of claim 16, wherein the absolute quantification is carried out by digital PCR.

18. A method of suppressing polymerase amplification activity inhibition effect by body fluid or a component thereof, encompassing the step of contacting the body fluid or the component thereof with a PVA sponge.

19. The method of claim 18, wherein the body fluid is blood or saliva.

20. A capturing agent or a capturing material that captures a microorganism or a portion of a microorganism, comprising a PVA sponge.

21.-63. (canceled)
